(19) United States
(12) Patent Application Publication
(10) Pub. No.: US 2020/0164006 A1
(43) Pub. Date: May 28, 2020

(54) METHODS AND COMPOSITIONS OF INCORPORATING A BIOACTIVE AGENT AND USE THEREOF

(71) Applicant: INNOVANUTRA INC., DAVIS, CA (US)

(72) Inventors: Anupama Tarakkad Ranganathan, Davis, CA (US); Mario Christopher Parks, Davis, CA (US); Stephen Allan Young, Davis, CA (US)

(73) Assignee: INNOVANUTRA INC., DAVIS, CA (US)

(21) Appl. No.: 16/612,756

(22) PCT Filed: May 20, 2018
(86) PCT No.: PCT/US2018/033560

§ 371 Date: Nov. 12, 2019

Related U.S. Application Data
(60) Provisional application No. 62/509,136, filed on May 21, 2017.

(51) Int. Cl. A61K 35/747; A61K 45/06; A61K 31/05; A61K 35/742; A61K 36/062; A61K 35/00

(52) U.S. Cl. CPC A61K 35/747; A61K 45/06; A61K 35/00; A61K 35/742; A61K 36/062; A61K 31/05

(57) ABSTRACT

Provided herein are compositions comprising a carrier and one or more bioactive agents in which one or more bioactive agents are incorporated into said carrier. Sugars that are naturally present with one or more bioactive agents as mixture are preferentially excluded out of said carrier in the composition. The compositions have wide applications including, but not limited to food and beverage industries. Also provided are methods of incorporating one or more bioactive agents into a carrier, compositions comprising a carrier and one or more bioactive agents and use of such methods and compositions, and kits. The methods are useful for (a) improving color, thermal, photo, and oxidative stability of bioactive agents, e.g., natural food colorants and other bioactive ingredients, (b) reducing and eliminating the usage of artificial chemicals such as sacrificial antioxidants and metal ion chelators that are used to improve bioactive stability.

### CROSS-REFERENCE TO RELATED APPLICATIONS

This application claims the priority under 35 U.S.C. § 119(e) to U.S. Provisional Patent Application Ser. No. 62/509,136 filed on May 21, 2017, which is incorporated by reference in its entirety.

### STATEMENT REGARDING FEDERALLY SPONSORED RESEARCH AND DEVELOPMENT

The invention was made with Government support under Small Business Innovation Research Grant Nos. 1549167 and 1660142 awarded by the National Science Foundation. The government has certain rights in the invention.

### FIELD OF THE INVENTION

Provided are methods, compositions of incorporating one or more bioactive agents in a carrier and use thereof.

### BACKGROUND OF THE INVENTION

The following description is provided to assist the understanding of the reader. None of the information provided or references cited is admitted to be prior art to the present invention.

Naturally occurring plant-based compounds that exhibit nutritional or health promoting properties as well as natural food colorants are usually associated with inherently low physio-chemical stability. Due to changes in food processing and storage, there may be a loss in nutrient value or fading of their natural color. They generally undergo an accelerated degradation with changes in temperature and pH or upon exposure to oxygen and/or light.

To address these challenges, the food and beverage processing industry currently uses sacrificial antioxidants, metal chelates or their combination with conventional incorporation approaches. The current solutions are sub-optimal, add substantial cost, often require the use of synthetic chemicals which are associated with a negative consumer perception and provide limited improvement in product shelf-life.

Different incorporation techniques based on physical, physio-chemical and chemical methods are used in the food industry. Many of these current incorporation systems provide limited thermal and oxidative stability, lack endogenous natural antioxidants and chelators, and can often only incorporate hydrophobic or hydrophilic compounds for incorporation.

### SUMMARY OF THE INVENTION

Provided herein are compositions, methods, and kits of incorporating one or more bioactive agents in one or more carriers and use thereof.

In one aspect, provided is a composition comprising (a) a carrier, (b) one or more bioactive agents, in which one or more bioactive agents are incorporated into the carrier. The one or more bioactive agents exists in nature in their unincorporated form as a mixture with sugars, and wherein sugars from the mixture are preferentially excluded from the formulation in the composition.

In some embodiments, the amount of sugar excluded from the carrier in the composition is at least about 10%, 15%, 25%, 30%, 33%, 35%, 40%, 45%, 50%, 55%, 60%, 65%, 70%, 75%, 80%, 85%, 90%, 95%, 98%, or 99% of the amount of sugar naturally present in a mixture with one or more bioactive agents.

In some embodiments, one or more bioactive agents are incorporated into the carrier in the presence of citric acid-sodium citrate at a pH of about 3-6. In some embodiments, the citric acid-sodium citrate buffer concentration can be about 0.001 M, 0.005 M, 0.075 M, 0.01 M, 0.0125 M, 0.025 M, 0.05 M, 0.075 M, 0.1 M, 0.125 M, 0.25 M, 0.5 M, 0.75 M, 1 M, 1.25 M, 1.5 M, 2 M, 2.5 M, 3 M, 3.5 M, or 4 M at a pH of about 3, 3.5, 4, 4.5, 5, 5.5, or 6.

In some embodiments, the one or more bioactive agents incorporated into the carrier of the composition have enhanced thermal stability, pH stability, color stability, oxidative stability, photo stability, or a combination of one or more stabilities as compared to unincorporated bioactive agents.

In some embodiments, the one or more bioactive agents incorporated into the carrier of the composition have enhanced thermal stability at a temperature range of about 4° C. to about 120° C., about 4° C. to about 100° C., about 10° C. to about 100° C., about 25° C. to about 100° C., about 10° C. to about 100° C., about 25° C. to about 100° C., about 25° C. to about 75° C. as compared to unincorporated bioactive agents. In some embodiments, the one or more bioactive agents incorporated into the carrier of the composition have enhanced thermal stability at temperatures of about 0° C., 4° C., 10° C., 15° C., 25° C., 30° C., 32° C., 35° C., 37° C., 40° C., 45° C., 50° C., 55° C., 60° C., 65° C., 75° C., 80° C., 90° C., 95° C., 100° C., 110° C., or 120° C. as compared to unincorporated bioactive agents.

In some embodiments, the one or more bioactive agents incorporated into the carrier of the composition have enhanced pH stability, stable at a pH range of about 2 to 10, about 3 to 10, about 3 to 6 as compared to unincorporated bioactive agents. In some embodiments, the one or more bioactive agents incorporated into the carrier of the composition have enhanced pH stability stable at a pH of about 2, 3, 4, 5, 6, 7, 8, 9, 10 as compared to unincorporated bioactive agents.

In some embodiments, the one or more bioactive agents incorporated into the carrier of the composition is less susceptible to free radicals as compared to unincorporated bioactive agents. In some embodiments, the one or more bioactive agents incorporated into the carrier of the composition is less susceptible to oxygen, hydroxyl, superoxides, peroxides, nitroxides or their radicals as compared to unincorporated bioactive agents.

In some embodiments, the one or more bioactive agents incorporated into the carrier of the composition are more resistant to degradation by UV or ambient light exposure as compared to unincorporated bioactive agents.

In some embodiments, the composition is a liquid, solid, gel or foam. In some embodiments, the composition is edible. In some embodiments, the composition is ice cream, gelato, mellorine, sherbet, sorbet, non-dairy desserts, yogurt and yogurt-based beverages, smoothies, kefir, gum, cakes, fruit juice, vegetable juice, coffee and tea beverages, bread, biscuits, chewing gum or confectionary. In some embodiments, the flavor or taste of said one or more bioactive agents incorporated into said carrier is masked as compared to unincorporated bioactive agents. In some embodiments, the taste or flavor is unpleasant. In some embodiments, the taste is sour, bitter, or astringent.

In some embodiments, the composition can be used safely by mammals. In some embodiments, the mammals are humans. In some embodiments, the composition can be used in cosmetics, personal care product, topical skin care, oral care, for antimicrobial applications, oral supplement, therapeutic applications. In some embodiments, the composition comprises a carrier and phytochemicals. In some embodiments, the phytochemicals have antioxidant properties. In some embodiments, the phytochemicals have colorants. In some embodiments, unpleasant tastes of the phytochemicals are masked in the composition. In some embodiments, the taste is sour, bitter, or astringent. In some embodiments, the cosmetics, personal care product, topical skin care, oral care can be foundation, lipsticks, cleansers, masks, exfoliants, blush, eye liner, eye shadow, lotions, creams, shampoos, toothpastes, tooth gels, mouth rinses, dental floss, tape or toothpicks.

In some embodiments, the composition further includes one or more protective agents incorporated into the carrier, in which one or more protective agents enhances the thermal stability, pH stability, color stability, oxidative stability, chemical stability, and/or photo stability of the one or more bioactive agents when co-incorporated with one or more bioactive agents into the carrier as compared to unincorporated bioactive agents.

In some embodiments, the composition can be administered orally, and wherein said one or more bioactive agents are protected from gastric juice during digestion. In some embodiments, the composition can be administered orally, and wherein one or more bioactive agents are preferentially delivered by the carrier in the small intestine. In some embodiments, the composition can be administered orally, and wherein one or more bioactive agents are preferentially by the carrier in the large intestine.

In some embodiments, the carrier of the composition can be a cell, and in which one or more bioactive agents improves the viability of the cell from exposure to heat, UV, ambient light, desiccation, pH, oxidative stress, or a combination thereof. In some embodiments, the cell can be a bacterial cell. In some embodiments, one or more bioactive agents that improves the viability of the cell comprises a phytochemical.

In some embodiments, the one of more bioactive agents is a phytochemical. A phytochemical may comprise polyphenols. In some embodiments, the polyphenol is a flavonoid such as anthocyanin, procyanidins, proanthocyanidin, catechins, epicatechins, prodelphinidins, flavonols, or a combination thereof.

In some embodiments, carriers of the above combination can be selected from a combinatorial platform. The combinatorial platform may comprise a choice of different carriers, e.g., a cell, cell aggregates with extracellular matrix, cell aggregates without extracellular matrix, cell wall particles, an extracellular membrane of a cell, ghost cell, spores, vegetable skin particles, fruit skin particles, plant tissue, animal tissue, or a virus.

In one aspect, provided is a method of incorporating one or more bioactive agents into a carrier. The method includes providing a carrier and one or more bioactive agents. The carrier in the presence of said one or more bioactive agents to 0.005M-4M buffer, pH 3-6, in which one or more bioactive agents are incorporated into the carrier. In some embodiments, In some embodiments, the citric acid-sodium citrate buffer concentration can be about 0.001 M, 0.005 M, 0.075 M, 0.01 M, 0.0125 M, 0.025 M, 0.05 M, 0.075 M, 0.1 M, 0.125 M, 0.25 M, 0.5 M, 0.75 M, 1 M, 1.25 M, 1.5 M, 2 M, 2.5 M, 3 M, 3.5 M, or 4 M at a pH of about 3, 3.5, 4, 4.5, 5, 5.5, or 6. In some embodiments, the carrier is subjected in the presence of one or more bioactive agents to vacuum pressure below 50% of absolute vacuum in which the bioactive agents are incorporated into the carrier. In one example, the carrier in the presence of the one or more bioactive agents is subjected to a vacuum pressure of about 0.1% to about 49% of absolute vacuum. In another example, the carrier in the presence of the one or more bioactive agents is subjected to a vacuum pressure of 0% of absolute vacuum. In varying embodiments, the carrier is subjected to vacuum pressure for less than about 30 minutes, e.g., less than about 25, 20, 15 or 10 minutes. In varying embodiments, the carrier is sealed in a container comprising below 50% of absolute vacuum levels, e.g., about 49%, 48%, 47%, 45%, 40%, 35%, 30%, 25%, 20%, 15%, 10%, 5%, 1%, or 0.1% of absolute vacuum levels. In varying embodiments, the carrier is subjected to multiple iterations, e.g., 2, 3, 4, 5, 6, 7, 8, 9, 10 iterations, of vacuum pressure. In varying embodiments, the methods further comprise, after subjecting the carrier to vacuum pressure, subjecting the carrier to positive external pressure. In some embodiments, the positive external pressure is at least about 30 MPa.

In one aspect, provided is a method of incorporating one or more bioactive agents into a carrier. The method includes providing a carrier and one or more bioactive agents. The carrier is subjected in the presence of the one or more bioactive agents to vacuum pressure of about 50% of absolute vacuum to about 99% of absolute vacuum in which the bioactive agents are incorporated into the carrier. In one example, the carrier in the presence of the one or more bioactive agents is subjected to a vacuum pressure of about: 0%, 0.1%, 1%, 2%, 3%, 4%, 5%, 6%, 7%, 8%, 9%, 10%, 11%, 12%, 13%, 14%, 15%, 16%, 17%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 55%, 60%, 65%, 66%, 67%, 68%, 70%, 75%, 80%, 85%, 90%, 95%, 98%, 99%, or 99% of absolute vacuum.

In one aspect, provided is a method of incorporating one or more bioactive agents into a carrier. The method includes providing one or more bioactive agents and a carrier. The carrier is subjected in the presence of the one or more bioactive agents to high concentration buffer at ambient temperature in which the bioactive agents are incorporated into the carriers. Examples of high concentration buffer include but are not limited to 4 M citric acid-sodium citrate, pH 3-6; 3 M citric acid-sodium citrate, pH 3-6; 2M citric acid-sodium citrate, pH 3-6; 1 M citric acid-sodium citrate, pH 3-6; 4 M acetic acid-sodium acetate pH 2.5-5.0; 3 M acetic acid-sodium acetate pH 2.5-5.0; 2 M acetic acid-sodium acetate pH 2.5-5.0; 1 M acetic acid-sodium acetate pH 2.5-5.0; 4 M citric acid-disodium hydrogen phosphate pH 2.5-5; 3M citric acid-disodium hydrogen phosphate pH 2.5-5; 2 M citric acid-disodium hydrogen phosphate pH 2.5-5; and 1 M citric acid-disodium hydrogen phosphate pH 2.5-5. In some embodiments, the buffer comprises salt, e.g., NaCl, sodium citrate, or sodium acetate at a concentration range of about 1 M to about 4 M. In some embodiments, the buffer includes non-ionic hydrophilic polymer at a concentration of about 2 to about 40 wt %. In some embodiments, the non-ionic hydrophilic polymer is selected from the group consisting of dextran, polyethylene glycol, and polyvinylpyrrolidone polymer.

In one embodiment, following the incorporation of bioactive agents into the carriers, the carriers are subjected to low concentration buffer to substantially remove unincorporated bioactive agents. Examples of low concentration buffers includes but are not limited to 0.001 M-0.05 M citric acid-sodium citrate, pH 3-6; 0.001 M-0.05 M citric acid-disodium hydrogen phosphate pH 2.5-4; and 0.001 M-0.05 M acetic acid-sodium acetate pH 3.5-4.0.

In one embodiment, the carrier in the presence of the one or more bioactive agents is subjected either a low or high concentration buffer at vacuum pressure of about 0% to about 99% of absolute vacuum. In one example, the carrier in the presence of the one or more bioactive agents is subjected to a vacuum pressure of about 0.1% to about 49% of absolute vacuum. In one example, the carrier in the presence of the one or more bioactive agents is subjected to a vacuum pressure of about: 0%, 0.1%, 1%, 2%, 3%, 4%, 5%, 6%, 7%, 8%, 9%, 10%, 11%, 12%, 13%, 14%, 15%, 16%, 17%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 55%, 60%, 65%, 66%, 67%, 68%, 70%, 75%, 80%, 85%, 90%, 95%, 98%, 99%, or 99% of absolute vacuum. In one example, the carrier in the presence of the one or more bioactive agents is subjected to a vacuum pressure of 0% of absolute vacuum.

In one aspect, provided are methods of enhancing the pH stability of one or more bioactive agents. The methods include incorporating the one or more bioactive agents into the carriers by any one of the methods disclosed herein in which incorporating the one or more bioactive agents into carrier enhances the pH stability of the one or more bioactive agents as compared to unincorporated bioactive agents. In one example, one or more bioactive agents is more stable at the pH range of: about 1 to about 10, about pH 2 to about pH 8, about pH 3 to about pH 6, about pH 3 to about pH 5 when incorporated into the carriers as compared to unincorporated bioactive agents. In one example, one or more bioactive agents is more stable at the pH of about: 1, 2, 3, 4, 5, 6, 7, 8, 9, 10, 11, 12, 13, or 14 when incorporated into the carriers as compared to unincorporated bioactive agents. In varying embodiments, the one or more bioactive agents incorporated into the carrier are chemically stable for at least 12 hours, e.g., at least about 12, 24, 72, 96 hours, 7 days, 8 days, 9 days, 10 days, 14 days, 21 days or more, e.g., at a temperature in the range of about 4° C. to about 65° C., e.g., about 4° C. to about 30° C., about 20° C.-55° C., about 25° C. to about 125° C. in a pH range of about 2-8. In varying embodiments, the carrier releases less than 25%, e.g., less than 20%, 15%, 10%, 5%, or less, of the incorporated bioactive agents at a pH range of about 2-8.

In one aspect, provided are methods of enhancing the thermal stability of one or more bioactive agents. The methods include incorporating one or more bioactive agents into the carriers by any one of the methods disclosed herein in which incorporating one or more bioactive agents into the carrier enhances the thermal stability of the one or more bioactive agents as compared to unincorporated bioactive agents. In one example, one or more bioactive agents is more stable at temperature range of: about 0° C. to about 250° C., about 25° C. to about 150° C., about 25° C. to about 80° C., or about 25° C. to about 75° C. when incorporated into the carriers as compared to unincorporated bioactive agents. In one example, one or more bioactive agents is more stable at temperature of: about: 20° C., 21° C., 22° C., 23° C., 24° C., 25° C., 26° C., 27° C., 28° C., 29° C., 30° C., 31° C., 32° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 70° C., 75° C., 80° C., 85° C., 90° C., 95° C., 100° C., 105° C., 110° C., 115° C., 120° C., 121° C., 125° C., 130° C., 150° C., 175° C., 200° C., 225° C., or 250° C. in a pH in the range of about pH 2-pH 8 for at least 12 hours, e.g., at least about 12, 24, 72, 96 hours, 7 days, 8 days, 9 days, 10 days, 14 days, 21 days when incorporated into the carriers as compared to unincorporated bioactive agents.

In one aspect, provided are methods of enhancing the oxidative stability of one or more bioactive agents. The methods include incorporating the one or more bioactive agents into the carriers by any one of the methods disclosed herein in which incorporating the one or more bioactive agents into carrier enhances the oxidative stability of the one or more bioactive agents as compared to unincorporated bioactive agents. In one example, one or more bioactive agents when incorporated into the carriers are less susceptible to peroxides, superoxides, nitroxides, or their radicals, or being oxidized at a pH range of about 2-8 across a range of temperatures of about 0° C. to about 200° C., about 20° C. to about 125° C., about 25° C. to about 65° C. for at least 12 hours, e.g., for at least about 12, 24, 72, 96 hours, 7 days, 8 days, 9 days, 10 days, 14 days, 21 days or more as compared to unincorporated bioactive agents.

In one aspect, provided are methods of enhancing the color stability of one or more bioactive agents. The methods include incorporating the one or more bioactive agents into the carriers by any one of the methods disclosed herein in which incorporating the one or more bioactive agents into a carrier enhances the color stability of the one or more bioactive agents as compared to unincorporated bioactive agents. In varying embodiments, the one or more bioactive agents incorporated into the carrier has enhanced color stability for at least 12 hours, e.g., at least about 12, 24, 72, 96 hours, 7 days, 8 days, 9 days, 10 days, 14 days, 21 days or more, e.g., at a temperature of about 0° C. to about 200° C., about 20° C. to about 125° C., about 25° C. to about 65° C., in a pH range of about 2-8 as compared to unincorporated bioactive agents.

In one aspect, provided are methods of enhancing the photo stability of one or more bioactive agents. The methods include incorporating the one or more bioactive agents into the carriers by any one of the methods disclosed herein in which incorporating the one or more bioactive agents into a carrier enhances the photo stability of the one or more bioactive agents as compared to unincorporated bioactive agents. In some embodiments, one or more bioactive agents have enhanced stability against UV rays while incorporated into one or more carriers as compared to unincorporated bioactive agents. In varying embodiments, the one or more bioactive agents incorporated into the carrier has enhanced photo stability for at least 12 hours, e.g., at least about 12, 24, 72, 96 hours, 7 days, 8 days, 9 days, 10 days, 14 days, 21 days, 30 days, 45 days, 60 days or more, e.g., at a temperature of about 0° C. to about 200° C., about 20° C. to about 125° C., about 25° C. to about 65° C., in a pH in the range of about 2-8 as compared to unincorporated bioactive agents.

In one aspect, provided are methods of selectively releasing one or more bioactive agents from a composition including a carrier and one or more bioactive agents in which one or more bioactive agents are incorporated into the carrier. The method includes subjecting the composition to a high concentration buffer. In some embodiments, the high concentration buffer is selected from the group consisting of 4 M citric acid-sodium citrate, pH 3, 3 M citric acid-sodium Citrate, pH 3, 2 M citric acid-sodium citrate, pH 3, and 1 M citric acid-sodium citrate, pH 3. In some embodiments, the buffer comprises sodium chloride, sodium citrate, or sodium acetate at a concentration range of about 50 mM to about super-saturated HO M). In some embodiments, the buffer includes non-ionic hydrophilic polymer at a concentration of about 2% to about 40% wt %. In some embodiments, the non-ionic hydrophilic polymer is selected from the group consisting of dextran, polyethylene glycol, and polyvinylpyrrolidone polymer. In some embodiments, the composition is produced by subjecting the carrier in the presence of one or more bioactive agents to vacuum. In some embodiments, the vacuum is about 0.1% to about 99%, about 0.1% to about 49% of absolute vacuum. In some embodiments, the vacuum is 0% of absolute vacuum.

In one aspect, provided are compositions comprising carrier and one or more bioactive agents in which one or more bioactive agents are incorporated into the carrier by the methods disclosed. In one embodiment, the composition is produced by subjecting the carrier in the presence of the one or more bioactive agents to vacuum pressure such that the bioactive agents are incorporated into the carrier. In some embodiments, the vacuum pressure is at least about 50% of absolute vacuum. In some embodiments, the vacuum pressure is below 50% of absolute vacuum. In some embodiments, the carrier in the presence of the one or more bioactive agents is subjected to a vacuum pressure of about 0.1% to about 49% of absolute vacuum. In some embodiments, the carrier in the presence of the one or more bioactive agents is subjected to 0% of absolute vacuum. In one example, the carrier in the presence of the one or more bioactive agents is subjected to a vacuum pressure of about: 0%, 0.1%, 1%, 2%, 3%, 4%, 5%, 6%, 7%, 8%, 9%, 10%, 11%, 12%, 13%, 14%, 15%, 16%, 17%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 55%, 60%, 65%, 66%, 67%, 68%, 70%, 75%, 80%, 85%, 90%, 95%, 98%, 99%, or 99% of absolute vacuum. In some embodiments, after subjecting the carrier in the presence of the one or more bioactive agents is subjected to a vacuum pressure, the carrier and one or more bioactive agents are further subjected to positive external pressure.

In some embodiments, the carrier can be a cellular aggregate with extracellular matrix, cellular aggregate without extracellular matrix, a ghost cell, an extra cellular membrane, vegetable skin particles, fruit skin particles, plant tissue, animal tissue, virus, spore, a colloid, or a micelle. In some embodiments, the cellular aggregates comprise extracellular matrix. In some embodiments, the cellular aggregates do not comprise extracellular matrix.

In some embodiments, the composition is edible by a mammal. In some embodiments, the edible composition is selected from the group consisting of a beverage, a food, vegetable extract, fruit extract, a nutraceutical, a compressed cake, a powder, a suspension, and a capsule. In some embodiments, the composition is inedible by a mammal. In some embodiments, the composition is applied to external surfaces of a mammal. In some embodiments, the composition is a part of a cosmetic formulation.

In some embodiments, the composition is stable to temperature from: about 0° C. to about 200° C., about 25° C. to about 150° C., about 25° C. to about 25° C. to about 125° C. In some embodiments, the composition is stable at temperature of about: 25° C., 30° C., 35° C., 37° C., 40° C., 45° C., 50° C., 55° C., 60° C., 65° C., 70° C., 75° C., 80° C., 85° C., 95° C., 100° C., 105° C., 110° C., 120° C., 121° C., 125° C., 130° C., 140° C., 150° C. for at least 1 hour, e.g., at least about 1, 2, 3, 4, 6, 12, 24, 72, 96 hours, 7 days, 8 days, 9 days, 10 days, 14 days, 21 days or more, in a pH in the range of about 2-8. In some embodiments, one or more bioactive agents of the composition is more stable at temperature of about: 25° C., 30° C., 35° C., 37° C., 40° C., 45° C., 50° C., 55° C., 60° C., 65° C., 70° C., 75° C., 80° C., 85° C., 95° C., 100° C., 105° C., 110° C., 120° C., 121° C., 125° C., 130° C., 140° C., 150° C. for at least 1 hour, e.g., at least about 1, 2, 3, 4, 6, 12, 24, 72, 96 hours, 7 days, 8 days, 9 days, 10 days, 14 days, 21 days or more, in a pH in the range of about 2-14 as compared to unincorporated bioactive agents.

In some embodiments, the composition further comprises one or more protective agents such that one or more protective agents enhances the thermal stability, pH stability, color stability, oxidative stability, chemical stability, and/or photo stability of the one or more bioactive agents as compared to unincorporated bioactive agents and incorporated bioactive agents without one or more protective agents.

In some embodiments, the composition further comprises a coating of biopolymer on the carrier, for example, chitosan coating on the carrier, after the incorporation of one or more bioactive agents into such a carrier. In some embodiments, the composition comprising a carrier, one or more bioactive agents, and optionally one or more protective coating agents are freeze-dried. In some embodiments, the carrier in the composition is a cell. In some embodiments, the carrier is a live cell. In some embodiments, the carrier is inactivated cell incapable of either reproduction or infecting another organism. In some embodiments, the carrier is an attenuated cell.

In one aspect, provided are methods for determining the incorporation conditions of one or more bioactive agents into one or more carriers. The methods include utilizing a combinatorial panel of carriers and one or more bioactive agents. The panel of carriers are subjected, in the presence of the one or more bioactive agents, to one or more conditions in which the bioactive agents are incorporated into said one or more carrier.

In one aspect, provided are methods for determining the of incorporation condition for a plurality of bioactive agents into a carrier. The methods include providing a plurality of bioactive agents and a carrier. The carrier is subjected in the presence of the plurality of bioactive agents to one or more conditions by which the bioactive agents are incorporated into the carrier. A condition suitable for incorporation of one or more bioactive agents into the carrier is selected based on the incorporation of one or more bioactive agents into the carriers. In some embodiments, selecting a condition suitable for incorporation is indicative of an optimal pre-treatment for one or more bioactive agents.

In some embodiments, selecting a condition suitable for incorporation is indicative of the best combination of carrier and one or more bioactive agents. In some embodiments, selecting a condition suitable for incorporation is indicative of an optimal buffer for incorporation. In some embodiments, selecting a condition suitable for incorporation is indicative of an optimal salt concentration for incorporation. In some embodiments, selecting a condition suitable for incorporation is indicative of an optimal solvent for one or more bioactive agents. In some embodiments, selecting a condition suitable for incorporation is indicative of an optimal vacuum pressure for incorporation.

In one aspect, provided are methods for topically administering or delivering a bioactive agent to a mammalian subject. The method includes contacting the skin or mucosal tissue of the mammalian subject with the composition disclosed above.

In another aspect, provided are methods of administering or delivering a bioactive compound to the intestine of a mammalian subject. The method includes orally administering to the mammalian subject the composition disclosed above. In one example, the intestine includes the small intestine. In another example, intestine includes the large intestine.

In another aspect, provided are methods of administering or delivering a bioactive compound to the colon of a mammalian subject. The method includes orally or rectally administering to the mammalian subject the composition disclosed above. In varying embodiments, the composition comprising the carrier and one or more bioactive agents binds to the intestinal tissue and releases the bioactive agents into the tissue. In varying embodiments, the release of the bioactive agents into the tissue are within about 30 min, 25 min, 20 min, 15 min, 10 min, 5 min, or less, of contacting or binding to the tissue. In some embodiments, one or more bioactive agents are preferentially delivered by the carrier of the composition in large intestine. In some embodiments, one or more bioactive agents are preferentially delivered by the carrier of the composition in small intestine. In some embodiments, one or more bioactive agents are protected from gastric juice during digestion. In some embodiments, one or more bioactive agents incorporated into the carrier of the composition are more bioavailable inside the gastrointestinal tract of a mammalian subject as compared to unincorporated bioactive agents. In some embodiments, one or more bioactive agents incorporated into the carrier of the composition are converted to a more bioavailable form after orally administering a mammalian subject with the composition

In some embodiments, the composition is produced by subjecting the carrier in the presence of the one or more bioactive agents and high concentration buffer to vacuum pressure in which the bioactive agents are incorporated into the carriers. In some embodiments, the carriers after incorporation of bioactive agents are subjected to low concentration buffer to substantially remove unincorporated bioactive agents.

In some embodiments, the carrier subjected to mechanical treatment prior to or during the introduction of one or more bioactive agents. In some embodiments, the mechanical treatment is selected from the group consisting of sonication, heat, and high pressure. In some embodiments, the carrier subjected to mechanical treatment prior to or during the introduction of one or more bioactive agents. In some embodiments, the chemical treatment is treatment with surfactant, solvent, enzyme, acid, base or a combination thereof.

In some embodiments, the carrier is subjected to the one or more bioactive agents in an aqueous solution. In some embodiments of the above aspects, the carrier is subjected to the one or more bioactive agents in a non-aqueous solution. In some embodiments of the above aspects, after subjecting the carrier in the presence of the one or more bioactive agents to vacuum pressure, the carrier and one or more bioactive agents is subjected to positive external pressure. In some embodiments of the above aspects, the carrier is subjected to multiple iterations of vacuum pressure in the presence of the one or more bioactive agents, e.g., 2, 3, 4, 5, 6, 7, 8, 9, 10 iterations, of vacuum pressure. In some embodiments, the carrier is subjected to multiple iterations of vacuum pressure and positive external pressure in the presence of the one or more bioactive agents. In some embodiments of the above aspects, the carrier is subjected to additional bioactive agents between each iteration of vacuum pressure.

In some embodiments, the carrier is chemically treated prior to subjecting the carrier in the presence of the one or more bioactive agents to vacuum pressure. In some embodiments, when the carrier in the presence of the one or more bioactive agents is subjected to vacuum pressure does not include heating and is performed at: an ambient temperature or at a temperature of about 0° C. to about 37° C. In some embodiments of the above aspects, the carrier in the presence of the one or more bioactive agents is subjected to vacuum pressure at a temperature of about 0° C. to about 200° C., 0° C. to about 150° C., 0° C. to about 125° C., 0° C. to about 95° C., or 0° C. to about 75° C. In some embodiments of the above aspects, the carrier in the presence of the one or more bioactive agents is subjected to vacuum pressure at a temperature of about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 40° C., 42° C., 45° C., 50° C., 55° C., 58° C., 60° C., 65° C., 70° C., 75° C., 80° C., 85° C., 90° C., 95° C., 100° C., 105° C., 110° C., 115° C., 120° C., 121° C., 125° C., 130° C., 150° C., 160° C., 175° C., 180° C., 190° C., or 200° C. In some embodiments, the carrier in the presence of the one or more bioactive agents is subjected to vacuum pressure in dark. In some embodiments of the above aspects, the carrier is incubated with the bioactive agent for a period of time prior to subjecting them to vacuum. In some embodiments of the above aspects, the carrier is incubated with the bioactive agent at a temperature of about 25° C. to about 125° C., about 25° C. to about 95° C., about 25° C. to about 75° C. prior to subjecting them to vacuum.

In some embodiments, the carrier in presence of one or more bioactive agents is subjected to a temperature of about 25° C. to about 125° C. prior to subjecting the carrier in presence of one or more bioactive agents to vacuum pressure. In some embodiments of the above aspects, the carrier in presence of one or more bioactive agents is subjected to a temperature of about 55° C. prior to subjecting the carrier in presence of one or more bioactive agents to vacuum pressure.

In varying embodiments, the carrier comprising one or more bioactive agents releases less than about 5% of the incorporated bioactive agents. In varying embodiments, the loading efficiency of the bioactive agent is at least about 15%, e.g., at least about 20%, 25%, 30%, 35%, 40%, 45%, 50%, 55%, 60%, 65%, 70%, 75%, 80%, 85%, or more.

In varying embodiments, the carrier with incorporated one or more bioactive agents can withstand pressures of at least about 100 MPa, temperatures of at least about 50° C., i.e., temperature of about 50° C., 55° C., 60° C., 65° C., 70° C., 75° C., 80° C., 85° C., 90° C., 95° C., 100° C., 105° C., 110° C., 120° C., 121° C., 130° C., 140° C., 150° C., 160° C., 175° C., 190° C., or 200° C., and a pH in the range of about 2 to about 10. In some embodiments, the carriers have an average or mean diameter in the range of about 0.03 μm to about 100 μm, e.g., in the range of about 0.1 μm to about 100 μm.

In some embodiments, the carrier is a cell, spore, cell aggregates with extracellular matrix, cell aggregates without extracellular matrix, cell wall particles, extracellular membrane, ghost cells, plant issue, animal tissue, tissue fragments, animal tissue homogenate, plant tissue homogenate, virus, a colloid, a micelle, fruit and vegetable skin particles (non-limiting examples include grape skin particles, cranberry skin particles, plum skin particles, tomato skin particles), fruits and vegetable particles (non-limiting examples include carrot particles, tomato particles, potato particles, prune particles, pea particles). In some embodiments, the cell is not viable. In some embodiments, the cell is inactivated. In some embodiments, the carrier is a live cell. In some embodiments, the carrier is inactive cell incapable or either reproduction or infecting other organism. In some embodiments, the carrier is an attenuated cell. In some embodiments, the cell includes a cell wall. In some embodiments, permeability of cell wall is modified. In some embodiments, the disulfide crosslinking between cell wall proteins are reduced. In some embodiments, the cells comprise an extracellular matrix. In some embodiments, the cellular aggregates do not comprise extracellular matrix.

In some embodiments, the carrier is a powder or a non-colloidal or a colloidal suspension. In some embodiments, the powder is powder of a plant product. Non-limiting examples of powder of a plant product include wheat flour, corn flower, barley flour, oat flower, rice flour, grape skin powder, carrot powder, dried fruit powder, seed powder, bark powder, root powder, lentil flour, and chick pea flour.

In some embodiments, the carrier is a prokaryotic cell. In some embodiments, the prokaryotic cell is a bacterial cell. In some embodiments, the bacterial cell is from gram negative bacteria. In some embodiments, the bacterial cell is from gram positive bacteria. Non limiting examples of bacterial cells include Lactobacillus paracasei, Lactobacillus acidophilus, Lactobacillus brevis, Lactobacillus casei, Lactobacillus fermentum, Lactobacillus gasseri, Lactobacillus helveticus, Lactobacillus plantarum, Lactobacillus reuteri, Lactobacillus rhamnosus, Lactobacillus sakei, Lactobacillus salivarius, Lactococcus lactis, Lactobacillus casei subsp. casei, Propionibacterium acidipropionici, Lactobacillus casei subsp. casei, Bifidobacterium bifidum, Bifidobacterium breve, Bifidobacterium infantis, Bifidobacterium lactis, Bifidobacterium longum, Pediococcus acidilactici, Pediococcus pentosaceus, Streptococcus thermophilus; Streptococcus sp., Actinomyces sp., Veillonella sp., Fusobacterium sp., Porphromonas sp., Prevotella sp., Treponema sp., Nisseria sp., Haemophilis sp., Eubacteria sp., Lactobacterium sp., Capnocytophaga sp., Eikenella sp., Leptotrichia sp., Peptostreptococcus sp., Staphylococcus sp., Propionibacterium sp., Streptococcus salivarius, Streptococcus gordonii, Streptococcus oralis, Streptococcus rattus, Streptococcus uberis, Streptococcus oligofermentans, Streptococcus sanguinis Streptococcus mitisStaphylococcus epidermidis, and Staphylococcus hominis. In some embodiments, the bacteria is an extremophile. Non-limiting examples of extremophiles include Deinococcus radiodurans, Deinococcus thermus, Deinococcus. aerius, Deinococcus. depolymerans, Deinococcus. deserti, Deinococcus. erythromyxa, Deinococcus. ficus, Deinococcus. frigens, Deinococcus. geothermalis, Deinococcus. gobiensis, Deinococcus. aerolatus, and Deinococcus. aerophilus. In some embodiments, incorporation of one or more bioactive agents into the bacterial cells enhances the thermal and photostability of said one or more bioactive agents.

In some embodiments, the carrier is non-pathogenic. In some embodiments, the carrier is harmless to mammals. In some embodiments, the carrier can be ingested by mammals. In some embodiments, the carrier is stable at pH 2-9. In some embodiments, the carrier is stable at stable at a temperature of about 0° C. to about 200° C. In some embodiments, the carrier is a bacterium. In some embodiments, the bacteria are harmless to mammals. In some embodiments, the bacteria can be ingested by mammals. In some embodiments, the bacteria can be intact in the gastrointestinal cavity of mammals.

In some embodiments, the carrier is a spore. In some embodiments, the spores are harmless to mammals. In some embodiments, the spores can be ingested by mammals. In some embodiments, the spores are from Lactobacillus sporogenes, Saccharomyces cerevisiae, Bacillus subtilis, Bacillus cereus, Bacillus clause, Bacillus pumilus, Bacillus laterosporus, Bacillus licheniformis, Paenibacillus polymyxa, Brevibacillus laterosporus, Sporolactobacillus dextrus, Sporolactobacillus inulinus, Sporolactobacillus laevis, Sporolactobacillus terrae and Sporolactobacillus vineae, Brevibacillus laterosporus, Paenibacillus polymyxa, Bacillus clausii, Bacillus pumilus, Bacillus subtilis, Penicillium camemberti, Penicillium roquefortii, Penicillium candidum, Rhizopus microsporus var. oligosporus, Rhizopus oryzae, Aspergillus oryzae, Aspergillus niger, Aspergillus sojae, Geotrichum candidum, Neurospora sitophila, Rhizomucor miehei, Monascus purpureus, Botrytis cinerea, Ustilago maydis, Agaricus bisporus, Pleurotus spp., Clitocybe nuda, Grifola frondosa, Morchella spp., Tricholoma matsutake, Lactarius spp., Hydnum repandum, Hericium erinaceus, Lentinus edodes, Flammulina velutipes, Hypsizygus tessulatus, Sparassis crispa, Volvariella volvacea, Fusarium venenatum, Tuber spp., Ganoderma lucidum and Ganoderma applanatum. In some embodiments, the spores are a mixture of spores from at least two organisms. In some embodiments, the spore is resistant to temperature range from about 0° C. to about 200° C., from about 10° C. to about 150° C., from about 25° C. to about 125° C. in a pH in the range of about 2-14.

In some embodiments, the carrier is a eukaryotic cell. In some embodiments, the eukaryotic cell is a plant cell or an algal cell. In some embodiments the algal cell is a Chlorophyta or a Chlorella. In some embodiments, the Chlorella cell is selected from Chlorella minutissima and Chlorella sorokiniana. In some embodiments, the cell is a fungal cell. In some embodiments, the cell is a yeast cell. Non-limiting examples of yeast cells are Saccharomyces cerevisiae, Saccharomyces fragilis, Saccharomyces pastorianus, Saccharomyces diastaticus, Saccharomyces exiguous, Saccharomyces bayanus, Dekkera (Brettanomyces) bruxellensis, Lachancea thermotolerans, Schizosaccharomyces pombe, Saccharomycodes ludwigii, Torulaspora delbrueckii, Candida utilis (torula yeast), Candida krusei, Candida stellate, Candida zemplinina, Debaromyces hansenii, Kluyveromyces marxianus, Kluyveromyces thermotolerans, Metschnikowia pulcherrima, Pichia fermentans, Pichia subpelliculosa, Rhodotorula mucilaginosa, Pichia anomala, Kloeckera apiculata, Zygosaccharomyces rouxii, Cyberlindnera jadinii, Lipomyces starkeyi, Dekkera anomala, Hanseniaspora uvarum, Hanseniaspora guilliermondii, Hansenula anomala, Issatchenkia orientalis, Isaatchenkia terricola, Phaffia rhodozyma and Yarrowia lipolytica. In some embodiments, the carrier is an animal cell.

In some embodiments, the carrier is an extracellular membrane of a cell or is from an extracellular membrane of a cell. In some embodiments, the carrier is a subcellular organelle of a cell or is from a subcellular organelle of a cell. In some embodiments, the subcellular organelle is selected from the group consisting of nucleus, a mitochondrion, chloroplast, Golgi body, nucleoid, microsome, vacuole, adiposome, cytoplasm and endoplasmic reticulum. In some embodiments, the carrier is an exosome or is from an exosome. In some embodiments, the carrier is an oil body or is from an oil body. In some embodiments, the carrier is a milk lipid globule or is from a milk lipid globule.

In some embodiments, the carrier is a virus. In some embodiments, the virus can infect a plant. In some embodiments, the virus can infect an animal. In some embodiments, the virus is a bacteriophage. In some embodiments, the virus is not viable. In some embodiments, the virus is inactivated. In some embodiments, the virus is harmless to mammals. In some embodiments, the virus can be ingested by mammals. In some embodiments, permeability of viral capsid is modified. In some embodiments, the virus comprises a lipid envelope.

In some embodiments, the carriers subjected to stress by treating with heat, pH or UV light, mechanical stresses, or a combination thereof prior to incorporation of one or more bioactive agents, and then utilizing the pre-stressed carriers for incorporation is another way to ensure enhancement in stability of the incorporated actives to changes in heat, pH or light. In some embodiments, the carriers are subjected to the exposure of UV light for about 30 seconds to about 10 minutes. In some embodiments, the carriers are subjected to the exposure of UV light for about 30 sec, 1 min, 1 min 30 sec, 2 min, 2 min 30 sec, 3 min, 3 min 30 sec, 4 min, 4 min 30 sec, 5 min, 5 min 30 sec, 6 min, 6 min 30 sec, 7 min, 7 min 30 sec, 8 min, 8 min 30 sec, 9 min, 9 min 30 sec, 10 min, 11 min, 12 min, 15 min, 20 min, 25 min, 30 min, or more. In some embodiments, the carriers are subjected to the exposure of pH of about 4 to about 12. In some embodiments, the carriers are subjected to the exposure of about 4, 4.5, 5, 5.5, 6, 6.5, 7, 7.5, 8, 8.5, 9, 9.5, 10, 10.5, 11, 11.5, 12.

In some embodiments, these pre-stressed carriers may be live or inactivated cells. In some embodiments, these pre-stressed carriers may be live or inactivated bacterial cells. In some embodiments, these pre-stressed carriers may be live or inactivated eukaryotic cells. In some embodiments, these pre-stressed carriers may be live or inactivated fungal cells such as yeast cells.

In some embodiments, one or more bioactive agents are hydrophobic molecules. In some embodiments, micelles, microemulsions, emulsions, nanoemulsions or colloidal formulations of hydrophobic molecules may be formulated using both volatile and non-volatile solvents. In some embodiments, the volatile solvent is removed by subjecting the carrier and one or more bioactive agents to vacuum, heat, mechanical stress, or a combination thereof. In some embodiments, the carrier and one or more bioactive agents are subjected to vacuum, heat, or a combination of vacuum and heat during the incorporation of one or more bioactive agents into the carrier. In some embodiments, the carrier and one or more bioactive agents are subjected to vacuum, heat, or a combination of vacuum and heat prior to the incorporation of one or more bioactive agents into the carrier. In some embodiments, the carrier either in the presence or in the absence of one or more bioactive agents is subjected to a temperature of about 25° C. to about 200° C., or about: 55° C., 60° C., 65° C., 70° C., 75° C., 80° C., 85° C., 95° C., 100° C., 110° C., 125° C., 130° C., 150° C., 175° C., or 200° C.

In some embodiments, one or more bioactive agents are hydrophilic molecules. In some embodiments, the bioactive agents are a mixture of hydrophobic and hydrophilic molecules. In some embodiments, one or more bioactive agents are hydrophilic in which the carrier in the presence of the one or more bioactive agents is subjected to a buffer comprising high salt concentration. In some embodiments, the salt concentration in the buffer is about 1 M to about 4 M sodium chloride, 1 M to 4 M sodium acetate, or 1 M to 4 M sodium citrate. In some embodiments, the salt concentration in the buffer is about 1 M, 1.5 M, 2 M, 2.5 M, 3 M, 3.5 M or 4 M sodium chloride, sodium acetate or sodium citrate. In some embodiments, one or more bioactive agents are hydrophilic and wherein the carrier in the presence of one or more bioactive agents is subjected to a buffer comprising non-ionic hydrophilic polymer. In some embodiments, the polymer concentration about 2-40 wt %. In some embodiments, the polymer is selected from the group consisting of dextran, polyethylene glycol, and polyvinylpyrrolidone polymer. In some embodiments, the polymer has a molecular weight of about 5000-13000 Da. In some embodiments, one or more bioactive agents are solubilized in acidified water.

In some embodiments, one or more bioactive agents are colorants. In some embodiments, the colorant is a naturally occurring colorant. Non-limiting examples of naturally occurring colorants include anthocyanins such as purple carrot extract, cranberry extract, grape skin extract, beet, betacyanins, betanins, betalains, carmine/cochineal, carotenoids, chlorophyll, spirulina, turmeric oleoresin, curcumin, annatto extract, beet extract, caramel extract, paprika oleoresin, or saffron extract. In some embodiments, the colorant is an artificial colorant. Non-limiting examples of artificial colorants include FD&C Blue No. 1, FD&C Blue No. 2, FD&C Green No. 3, FD&C Red No. 3, FD&C Red No. 40, FD&C Yellow No. 5, FD&C Yellow No. 6, Orange B, and Citrus Red No. 2, a fluorescein dye, a rhodamine dye, a coumarin, a pyrene dye, a xanthene dye and an azo dye.

In some embodiments, the bioactive agents are edible by mammals. In some embodiments, the bioactive agents are a plant product, plant extract, fruit juice, fruit concentrate, vegetable juice, vegetable concentrate or a mixture thereof. In some embodiments, the plant product, plant extract, fruit juice or fruit concentrate, vegetable juice or vegetable concentrate is from cranberry, strawberry, raspberry, blackberry, blueberry, grape, purple carrot, acai fruits, aronia, black and red currents, bilberry, boysenberry, yumberry, elderberry, goji berry, grapefruit, guava, kiwi fruit, lemon, lime, nectarine, papaya, passion fruit, peach, pear, pineapple, peas, plum, lychee, mango, mangosteen, sweet potato, watermelon, spinach, soursop, sea buckthorn, raisin, prune, black tomato, blood orange, blackthorn, bog bilberry, cloudberry, crowberry, hibiscus, lingonberry, magellan barberry, maqui berry, mountain bilberry, red gooseberry, red grape, rowanberry, sour cherry, sweet cherry, tayberry, annatto, pomegranate, paprika, saffron, red cabbage, broccoli, kale, squash, celery, cucumber, jicama, orange carrot, onion, pumpkin, capers, red beet, rhubarb, tea, coffee, cocoa, cacao, guarana, guayusa, rooibos, or yerba mate or a mixture thereof. In some embodiments, the plant extract may be prepared from roots, stems, leaves, seeds, fruits and flowers of edible plants.

In some embodiments, one or more bioactive agents exists in nature in their unincorporated form as a mixture with sugars. In some embodiments, the sugars from one or more bioactive agents is a monosaccharide (e.g., glucose, fructose), disaccharide (e.g., sucrose), or oligosaccharides, or a mixture thereof.

In some embodiments, one or more bioactive agents have a sour, bitter, or astringent taste.

In some embodiments, the compositions comprising a carrier and one or more bioactive agents in which one or more bioactive agents are incorporated into the carrier are edible by mammals. In some embodiments, the bioactive agent and/or the edible composition is selected from the group consisting of a beverage, a food, vegetable extract, fruit extract, a nutraceutical, ice cream, gelato, mellorine, sherbet, sorbet, non-dairy desserts, yogurt and yogurt-based beverages, kefir, chewing gum, cakes, cookies, smoothies, fruit juice, vegetable juice, bread, biscuits, probiotics, confectionary and a capsule. In some embodiments, the compositions comprising carrier and one or more bioactive agents in which one or more bioactive agents are incorporated into the carrier by the methods disclosed above are mixed with food.

In some embodiments, the bioactive agents can comprise a small organic compound, a peptide, a polypeptide, a polynucleotide, and/or a fatty acid. In some embodiments, the bioactive agents can be small organic compound, a peptide, a polypeptide, a polynucleotide, and/or a fatty acid. In some embodiments, one or more bioactive agents have a molecular weight in the range of about 10 Da to about 30 kDa. In some embodiments, at least one of the one or more bioactive agents is a small organic compound. In some embodiments, the small organic compound is solubilized or suspended in an aqueous solution comprising a lower alcohol.

In some embodiments, the small organic compound is selected from the group consisting of curcuminoid, turmeric, a steroid, a saponin, a betalain, carminic acid, carmine, a flavonoid, a retinoid, a vitamin, a flavorant, a colorant, a dye, a pesticide, an herbicide, a fungicide, an antioxidant and a chemotherapeutic agent.

In some embodiments, the small organic compound is selected from the group consisting of a phenolic acid, a flavonoid, a terpenoid, an alkaloid, a phytosterol, a lipid-soluble vitamin, a water-soluble vitamin, a bioactive lipid, a stilbenoid, a coumarin, a lignoid, a xanthonoid, a glycoside, an anthraquinone, and mixtures thereof. In some embodiments, the phenolic acid is selected from the group consisting of a hydroxybenzoic acid, a hydroxycinnamic acid, and derivatives thereof. In some embodiments, the phenolic acid is a hydroxybenzoic acid derivative selected from the group consisting of p-hydroxybenzoic acid, gallic acid, protocatechuic acid, vanillic acid and syringic acid. In some embodiments, the phenolic acid is a hydroxycinnamic acid derivative selected from the group consisting of p-coumaric acid, caffeic acid, ferulic acid, curcurmin, chlorogenic acid and sinapic acid.

In some embodiments, the flavonoid is selected from the group consisting of flavonols (fisetin, quercetin, kaempferol, myricetin, and galangin), flavones (luteolin, apigenin, and chrysin), flavanols (catechin, epicatechin, epigallocatechin (EGC), epicatechin gallate (ECG), and EGC gallate (EGCG)), flavanones (naringenin, hesperitin, and eriodictyol), biflavanoids (isocryptomerin and amentoflavone), proanthocyanidins, anthocyanidins and/or anthocyanins (cyanidin, malvidin, peonidin, pelargonidin, and delphinidin), isoflavonoids (genistein, daidzein, glycitein, and formononetin), chalcones (isobavachalcone, kanzonol C, erioschalcones A and B, and panduratin C), quinones, xanthones, acridones, kalihinanes, artemisinin and its derivatives, quinine and its derivatives, and mixtures thereof.

In some embodiments, the terpenoid is selected from the group consisting of carotenoids (lycopene, lutein, zeaxanthin, β-carotene, β-cryptoxanthin, retinol and its derivatives), saponins (ginsenoside, astragaloside, and phanoside), terpenoid acids (dehydrotrametenolic acid), and mixtures thereof.

In some embodiments, the alkaloid is the alkaloid is selected from the group consisting of β-carbolines (nostocarboline, manzanine A, and homofascaplysin), xanthines (caffeine, theophylline, and theobromine), phenethylamines (dopamine, epinephrine, and norepinephrine), quinolones (berberine, protopine, and β hydrastine), isoquinolines (schulzeines A, B and C) carbazoles (mahanimbine), bis-benzylisoquinolines (fangachinoline, tetrandine and stephenanthrine), quinolizidines (lupanine and 2-thionosparteine), and mixtures thereof.

In some embodiments, the phytosterol is selected from the group consisting of sitosterol (3β-stigmast-5-en-3 ol), sitostanol (3β,5α-stigmastan-3-ol), campesterol (3β-ergost-5-en-3-ol), campestanol (3β,5α-ergostan-3-ol), stigmasterol (3β-stigmasta-5,22-dien-3-ol) and brassicasterol (3β-ergosta-5,22-dien-3-ol).

In some embodiments, the lipid-soluble vitamin is selected from the group consisting of vitamin A (retinol, beta-carotene), retinal, retinoic acid, retinyl esters (e.g., retinyl acetate, retinyl palmitate and retinyl propionate) and provitamin A carotenoids (e.g., beta-carotene, alpha-carotene and beta-cryptoxanthin), vitamin E, vitamin D and vitamin K.

In some embodiments, the water-soluble vitamin is selected from the group consisting of vitamin C, B vitamins (B-1, B-2, B-3, B-6, B-7, B-9, B-12, B10 or coenzyme b10), nicotinic acid, niacinamide, nicotinamide, and 5-methyltetrahydrofolate (5-MTHF).

In some embodiments, the small organic compound is a flavorant selected from the group consisting of diacetyl (buttery), isoamyl acetate (banana), benzaldehyde (bitter almond), cinnamic aldehyde (cinnamon), ethyl propionate (fruity), methyl anthranilate (grape), limonene (orange), ethyl decadienoate (pear), allyl hexanoate (pineapple), ethyl maltol (cotton candy), ethylvanillin (vanilla), methyl salicylate (wintergreen), 2-methyl-2-pentenoic acid (fresh strawberry), 2-methyl-4-pentenoic acid (cooked strawberry), menthol, glutamic acid, glycine, guanylic acid, inosinic acid, a 5′-ribonucleotide salt, acetic acid, ascorbic acid, citric acid, fumaric acid, lactic acid, malic acid, phosphoric acid, tartaric acid and mixtures thereof.

In some embodiments, the small organic compound is a vitamin selected from the group consisting of retinol, retinal, retinoic acid, retinyl esters (e.g., retinyl acetate, retinyl palmitate and retinyl propionate) and provitamin A carotenoids (e.g., beta-carotene, alpha-carotene and beta-cryptoxanthin), retinol (vitamin A), thiamine (vitamin B1), riboflavin (vitamin B2), niacin, pyridoxine HCl (vitamin B6), folate, cyanocobalamin (vitamin B12), biotin, pantothenic acid, vitamin C, vitamin D (including cholecalciferol (D2) and ergocalciferol (D3)), vitamin E, vitamin K, and mixtures thereof.

In some embodiments, the small organic compound is a chemotherapeutic agent selected from the group consisting of alkylating agent(s), stimulant(s), platinum-coordination complex(es), anti-metabolite(s), plant alkaloid(s) and/or terpenoid(s), vinca alkaloid(s), podophyllotoxin(s), camptothecin(s), anthracycline(s), aromatase inhibitor(s), taxane(s), topoisomerase inhibitor(s), antibiotic(s), hormone(s), differentiating agent(s), kinase inhibitor(s), anti-inflammatory compounds, and antineoplastic agent(s).

In some embodiments, the carrier comprises one or more members of a binding pair on its external surface. Non-limiting examples of members of a binding pair antigen/antibody, hormone/hormone receptor receptors, biotin/avidin.

In some embodiments, one or more bioactive agents are incorporated inside the carrier. In some embodiments, one or more bioactive agents are adsorbed on the surface of the carrier. In some embodiments, the carrier and one or more bioactive agents are members of a binding pair.

In some embodiments, provided are methods for enhancing the color stability, photo stability, pH stability, oxidative stability, chemical stability and/or heat stability of one or more bioactive agents. The methods include incorporating one or more bioactive agents into the carriers in the presence of high salt buffer such that incorporating one or more bioactive agents in the presence of high salt buffer into a carrier enhances the color stability, pH stability, photo stability, and/or heat stability of the one or more bioactive agents as compared to unincorporated bioactive agents. Non-limiting examples of high concentration buffers includes 4 M citric acid-sodium citrate, 2 M citric acid-sodium citrate, and 1 M citric acid-sodium Citrate, at a pH of about 3-5.

In varying embodiments, the one or more bioactive agents incorporated into the carrier has enhanced color stability, photo stability, pH stability, oxidative stability, chemical stability and/or heat stability for at least 12 hours, e.g., at least about 12, 24, 72, 96 hours, 7 days, 8 days, 9 days, 10 days, 14 days, 21 days, 30 days, 45 days, 60 days or more, e.g., at a temperature of about 0° C. to about 200° C., about 20° C. to about 125° C., about 25° C. to about 65° C., in a pH in the range of about 2-8 as compared to unincorporated bioactive agents.

In some embodiments, the methods further include co-incorporating one or more protective agents with one or more bioactive agents into the carrier in the presence of high salt buffer such that co-incorporation of one or more protective agents with one or more bioactive agents into the carrier enhances the color stability, photo stability, pH stability, oxidative stability, chemical stability and/or heat stability of the one or more bioactive agents as compared to unincorporated bioactive agents and incorporated bioactive agents without said one or more protective agents. In some embodiments, the methods further include coating one or more carriers with biopolymers after the incorporation of one or more bioactive agents such that coating with the biopolymer enhances the color stability of said one or more bioactive agents.

In some embodiments, one or more protective agents enhances the oxidative stability of one or more bioactive agents as compared to unincorporated bioactive agents and incorporated bioactive agents without one or more protective agents. Non-limiting examples of such protective agents include anti-oxidants, vitamin C, vitamin A, vitamin E, glutathione, uric acid, melatonin, thioredoxin, propyl gallate (PG, E310), tertiary butylhydroquinone (TBHQ), butylated hydroxyanisole (BHA, E320) butylated hydroxytoluene (BHT, E321), EDTA, EGTA, and plant derived antioxidants.

Non-limiting examples of plant-derived antioxidants include rosemary, cinnamon, cassia, thyme, basil, oregano, clove, lemongrass, lavender, tarragon, lemon balm, dill, parsley, cilantro, sage, marjoram, bay laurel, dandelion, mugwort, ginseng, cardamom, black pepper, burdock, mustard, savory, nutmeg, chicory, caraway, chervil, fenugreek, pepper, celery, epazote, horseradish, wasabi, ginger, rue, sorrel, goldenrod, valerian, horehound, hibiscus, echinacea, walnut, juniper, artichoke, arugula, cardoon, holy basil, milk thistle, allspice, anise, licorice, aloe, garlic, black cohosh, witch hazel, saw palmetto, pandan, paprika, watercress, perilla, orris, vanilla, kawakawa, mint, peppermint, spearmint, wintergreen, catnip, sumac, poppy, safflower, saffron, hops, yarrow, cinchona, quassia, turmeric, lemon myrtle, lemon verbena, citrus peel and leaf including orange, lemon, grapefruit, tangerine, kumquat, kaffir, bergamot and bitter orange, pomegranate peel, olive extracts, and a combination of one or more of the above.

In some embodiments, one of more protective agents enhances the heat stability of one or more bioactive agents as compared to unincorporated bioactive agents and incorporated bioactive agents without said one or more protective agents. Non-limiting examples of such protective agents include heat shock proteins and amino acids such as arginine, proline, histidine.

In some embodiments, one of more protective agents enhances the pH stability of one or more bioactive agents as compared to unincorporated bioactive agents and incorporated bioactive agents without said one or more protective agents. Non-limiting examples of such protective agents include amino acids such as arginine, proline, histidine.

In some embodiments, one of more protective agents enhances the color stability of the one or more bioactive agents as compared to unincorporated bioactive agents and incorporated bioactive agents without said one or more protective agents. Non-limiting examples of such protective agents include organic acids such as rosmarinic acid, ursolic acid, ferulic acid, vanillic acid.

In some embodiments, one or more bioactive agents are polynucleotides and one or more protective agents are nuclease inhibitor. Non-limiting examples of nuclease inhibitor include EDTA, EGTA, RNaseIn™ (Promega Corporation, USA). In some embodiments, one or more bioactive agents are polypeptides and one or more protective agents are protease inhibitor. Non-limiting examples of protease inhibitor include trypsin inhibitor.

In some embodiments, one or more carriers are coated with biopolymers after the incorporation of one or more bioactive agents such that coating with the biopolymer enhances the color stability of one or more bioactive agents. Non-limiting example of such biopolymer include chitosan.

In some embodiments, one or more bioactive agents incorporated into a carrier of a composition can be extracted from the carrier in the presence of 5-100% ethanol, dimethyl sulfoxide, 5-100% methanol, acidified methanol and 0.005 M to 4 M citric acid-sodium citrate buffer at a pH of about 3-6.

In one aspect, a combinatorial platform approach for the incorporation of different bioactive agents using varying conditions and formulations may be used. FIG. 1B shows the incorporation of a red-colored fruit extract that may result in different formulations of varying stability. The formulation with the best physio-chemical stability is used for testing in a finished product. A good red color retention is observed when the extract is incorporated using the formulation of the present methods disclosed as compared to unincorporated products.

In one aspect, provided are kits for determining the incorporation condition of one or more bioactive agents into one or more carriers. The kits include one or more carriers, one or more bioactive agents, and instructions for incorporating said one or more bioactive agents into said one or more carriers.

In one aspect provided are kits for determining the enhanced heat, pH, oxidative, chemical, and/or photostability of one or more bioactive agents while incorporated into one or more carriers. The kits include one or more bioactive agents incorporated into one or more carriers, buffers, and instructions for testing the stability of one or more bioactive agents to heat, pH, oxidation, chemicals and/or light.

In some embodiments, the kits further include one or more protective agents in which one or more protective agents enhances the thermal stability, pH stability, color stability, oxidative stability, chemical stability, and/or photo stability of the one or more bioactive agents as compared to unincorporated bioactive agents and incorporated bioactive agents without said one or more protective agents.

### BRIEF DESCRIPTION OF FIGURES

FIG. 1. FIG. 1A shows an exemplary schematic of the incorporation process of bioactive agents. FIG. 1B shows an exemplary schematic combinatorial platform approach for the incorporation of different bioactive agents using varying formulations and carriers.

FIG. 2. FIG. 2A shows the incorporated cranberry extract in L. casei cells (sample) and L. casei cells without cranberry extract (control). FIG. 2B shows the result of a comparison of the stability of the incorporated with the unincorporated cranberry extracts at pH 3, 4 and 5. FIG. 2C shows the percent color retention of unincorporated cranberry extract. FIG. 2D shows the percent color retention of L. casei—incorporated cranberry extract, respectively, over different time points at different pHs 3, 4, 5 and 6. FIG. 2E shows a comparison of unincorporated (left) and L. casei—incorporated cranberry extract (right). The vials were stored for ˜2 months, in (a) pH 3, (b) pH 4, (c) pH 5.

FIG. 3. FIG. 3A shows a standard calibration curve for quercetin in dimethyl sulfoxide (DMSO). Absorbance at 388 nm for varying concentration of quercetin in DMSO were plotted. FIG. 3B shows multi-photon fluorescence imaging of quercetin incorporated in yeast using different buffers, (a) 0.1 M phosphate buffer, pH 5.8, (b) 0.1 M citric acid-sodium citrate buffer, pH 3, (c) 0.1 M citric acid-sodium citrate buffer, pH 6. The excitation was 760 nm and emission DAPI 433/24, Objective: 63× Magnification: 66.15×. FIG. 3C shows pH stability data for quercetin incorporated in yeast (left bar), L. casei (middle bar) and unincorporated quercetin (right bar) when exposed to citric acid/sodium citrate buffer at pH 3 and 6 at various time points. FIG. 3D shows oxidative stability data for quercetin incorporated in yeast, with and without free radical generator AAPH (AAPH (2,2′-azobis(2-amidinopropane) dihydrochloride. induced oxidation. PBS used in the control experiment instead of AAPH.

FIG. 4. FIG. 4A shows a qualitative comparison of various carriers (yeast, L. sporogenes and L. casei) for color stability of water-soluble purple carrot extracts. FIG. 4B shows the color stability of purple carrot extract incorporated in L. paracasei at pH 3-6 (indicated on the bottle) at different time points using citric acid-sodium citrate buffers at room temperature.

FIG. 5. FIG. 5A shows a calibration curve for retinol in 100% methanol. Absorbance at 325 nm for varying concentration of retinol in 100% methanol were plotted. FIG. 5B shows the comparative oxidative stability data for retinol as % retention of retinol incorporated in yeast (green), L. casei (blue) and unincorporated retinol (red). FIG. 5C shows a comparison of performance of carriers (yeast and L. casei) for improved oxidative stability of retinol.

FIG. 6. FIG. 6A shows the enhanced color stability of purple carrot extract at different pH after co-incorporation with rosmarinic acid. FIG. 6B shows comparison of color stability of purple carrot extract in incorporated formulations versus the unincorporated purple carrot extract in chewing gum products for 4 weeks. FIG. 6C shows comparison of color stability of purple carrot extract in various incorporated formulations versus unincorporated formulations in chewing gum products for 2 weeks. FIG. 6D shows the color stability of incorporated purple carrot extract and rosmarinic acid and unincorporated purple carrot extract in chewing gum products after subjecting to heat (approximately 100° C.) and subsequent incubation at 37° C.

FIG. 7. FIG. 7 shows a comparison of color stability of incorporated purple carrot extract in L. paracasei, in different buffers: (a) low concentration citric acid-sodium citrate, (b) high concentration citric acid-sodium citrate (c) unincorporated purple carrot added to chewing gum with a low concentration citric acid-sodium citrate, pH 3 buffer as a medium, (d) unincorporated purple carrot added to chewing gum with a high concentration citric acid-sodium citrate, pH 3 buffer as a medium.

FIG. 8. FIG. 8A shows a calibration curve of gallic acid in water. FIG. 8B shows an exemplary schematic showing the incorporation of phenolics into carriers (e.g., microbial cells) after addition of fruit/vegetable concentrate into carriers (e.g., microbial cells) and the formation of incorporated carriers (e.g., microbial cells) with the supernatant containing the unincorporated or unbound phenolics.

FIG. 9. FIG. 9 shows comparative bar charts showing % yields of total phenolics obtained by the Folin-Ciocalteau assay after passive and vacuum-based infusion of cranberry concentrate in L. paracasei cells. % Yield=amount of phenolics incorporated in cells/original mass of microbial cells taken for incorporation.

FIG. 10. FIG. 10 shows the percent yields of phenolics in different buffer concentrations with L. paracasei and Candida utilis (torula yeast) containing cranberry (FIGS. 10A and 10B, respectively) and strawberry concentrate (FIGS. 10C and 10D, respectively). Each bar chart also depicts the type of incorporation buffer and wash buffer used in each infusion. % Yield=Amount of phenolics in cells/Original mass of microbial cells taken for incorporation.

FIG. 11. FIG. 11 shows the percent yields of phenolics in different buffer concentrations with L. paracasei and torula yeast containing raspberry (FIGS. 11A and 11B, respectively) and blackberry concentrate (FIGS. 11C and 11D, respectively). Each bar chart also depicts the type of incorporation buffer and wash buffer used in each infusion. % Yield=Amount of phenolics in cells/Original mass of microbial cells taken for incorporation.

FIG. 12. FIG. 12 shows the percent yields of phenolics in different buffer concentrations with L. paracasei and torula yeast containing grape (FIGS. 12A and 12B, respectively) and purple carrot concentrate (FIGS. 12C and 12D, respectively). Each bar chart also depicts the type of incorporation buffer and wash buffer used in each infusion. % Yield=Amount of phenolics in cells/Original mass of microbial cells taken for incorporation.

FIG. 13. FIG. 13 shows tables with Brix values (degree) shown for the stock, supernatant and washes for formulations with L. paracasei and torula yeast containing cranberry (A and B) and strawberry concentrate (C and D). The stock solution refers to what is added to the cells prior to incorporation. This is equal to 1 mL of the fruit concentrate+4 mL of buffer, which gives a Brix value of 10 degree for cranberry and ˜15 degree for the other fruit concentrates. The sum of the Brix values for the supernatant and washes is equivalent to that of the stock solution.

FIG. 14. FIG. 14 shows tables with Brix values (degree) shown for the stock, supernatant and washes for formulations with L. paracasei and torula yeast containing raspberry (A and B) and blackberry (C and D) and grape concentrates (E and F). The stock solution refers to what is added to the cells prior to incorporation. This is equal to 1 mL of the fruit concentrate+4 mL of buffer, which gives a Brix value of 10 degree for cranberry and 15 degree for the other fruit concentrates. The sum of the Brix values for the supernatant and washes is equivalent to that of the stock solution.

FIG. 15. FIG. 15 shows the results of taste masking experiments with incorporated and unincorporated fruit concentrates in 5 g of yogurt. FIG. 15A Left to right: incorporated cranberry extract in L. paracasei (0.15 g), unincorporated cranberry extract (0.1 g), unincorporated cranberry extract (0.2 g), unincorporated cranberry extract (0.3 g), each in 5 g of yogurt. FIG. 15B Left to right: incorporated cranberry extract in P. acidilactici (0.15 g), unincorporated cranberry extract (0.1 g), unincorporated cranberry extract (0.2 g), unincorporated cranberry extract (0.3 g), each in 5 g of yogurt. In each case, unpleasant taste ranking refers to either sourness, bitterness or astringency.

FIG. 16. FIG. 16A shows the pictures of biscuits with formulations of cranberry extract (CE) in L. paracasei added to biscuit or various concentrations of unincorporated CE added to biscuit. A1: no CE, A2: 0.5 g unincorporated CE in 10 g biscuit; A3: 0.8 g unincorporated CE in 10 g biscuit, A4: 0.3 g of formulation of CE incorporated in L. paracasei in 10 g of biscuit. FIG. 16B shows a table indicating the color, initial flavor, and aftertaste of biscuits comprising CE incorporated in L. paracasei or unincorporated CE.

FIG. 17. shows a table indicating the color, initial flavor, and aftertaste of Kefir comprising CE incorporated in L. paracasei or unincorporated CE.

FIG. 18. FIG. 18 shows the results of the cell viability studies after incubating overnight at various concentrations of citric acid-sodium citrate buffer. FIG. 18A shows the negative controls without the incorporated fruit concentrates, on MRS Agar plates. The cells were incubated with the corresponding citric acid sodium citrate buffer (pH 3). FIG. 18B shows the results wherein bacterial cells were incubated with blackberry extract and corresponding citric acid sodium citrate buffer (pH 3) conditions. TNTC refers to colonies Too Numerous To Count.

FIG. 19. FIG. 19 shows a schematic illustrating the preferential infusion of antioxidants or phenolics into a composition and preferentially excluding the sugars from the starting mixture.

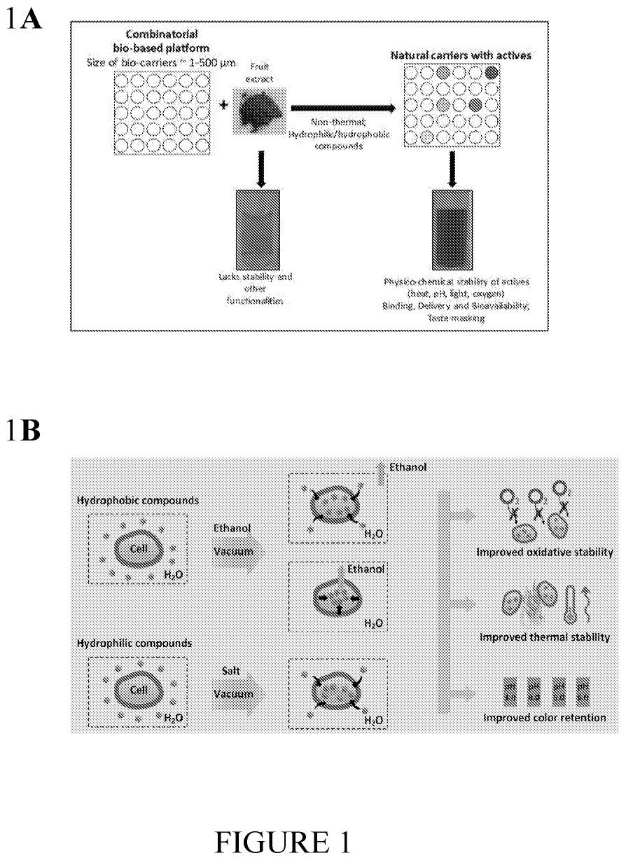

### DETAILED DESCRIPTION OF THE INVENTION

Provided herein are compositions comprising a carrier and one or more bioactive agents, methods of incorporating one or more bioactive agents into a carrier, and use of such methods and compositions.

The inventors have devised an efficient method to incorporate one or more bioactive agents into a carrier from a mixture of one or more bioactive agents with inherent sugars, wherein sugars from the mixture are preferentially excluded out of the carrier and preferentially not incorporated into the carrier. The compositions and methods disclosed have applications in a wide range of industries including food and beverage, cosmetic, cosmeceutical, dental and pharmaceutical industries.

In some embodiments, the methods include using a high concentration buffer to incorporate the ingredients and a low concentration buffer to wash and remove the unincorporated ingredients. The inventors significantly improved the bioactive loading or incorporation yields of hydrophilic extracts in the presence of specific buffers with simple passive infusion or utilizing vacuum or multiple vacuum infusions.

This application provides methods for (a) improving color, pH, thermal, photo- and oxidative stability of natural food colorants and other bioactive ingredients, (b) eliminating the usage of artificial chemicals such as sacrificial antioxidants and metal ion chelators that are used to improve bioactive stability. This application provides improved processing methods that can provide highly efficient and rapid incorporation of one or more bioactive agents into a carrier without the requirement of elevated temperatures. In this regard, the process can enable translation of the cell-based incorporation process to industrial practice.

The methods disclosed in this application provides a solution to the food and beverage and nutraceutical industries by enhancing the thermal, pH, and color stability of bioactive agents while incorporated into the carrier.

As used herein, the term “bioactive agent” refers to small organic compounds, colorants, polypeptides (e.g., ligands, antibodies), peptidomimetics, nucleic acids, plant products, plant extracts, fruit extracts, fruit juice, fruit concentrates, vegetable extracts, vegetable concentrates and the like, that can be incorporated in the carrier described herein. In some examples, the bioactive agent can be adsorbed by the carrier. In some embodiments, plant extracts, fruit extracts, fruit juice, fruit concentrates, vegetable extracts, vegetable concentrates comprise small organic compounds, colorants, antioxidants, polypeptides. In some embodiments, one or more bioactive agents can be a polyphenol. In some embodiments, such polyphenol can be a flavonoid, phenolic acids, or a combination thereof.

As used herein the terms “incorporating”, “incorporated”, “encapsulating”, and “encapsulated”, “infusion”, “infused” are all used interchangeably in the context of a carrier. The terms mean associating, adsorbing, binding or present inside a carrier, the latter akin to a host-guest complex. In some embodiments, incorporated into a carrier in the context of one or more bioactive agents means that one or more bioactive agents are included inside a carrier. Non-limiting examples include inside a cell, a cell aggregate, or a spore.

As used herein the term preferentially excluded out of a carrier in the context of sugars means that at least about 10%, 15%, 25%, 30%, 35%, 40%, 45%, 50%, 55%, 60%, 65%, 70%, 75%, 80%, 85%, 90%, 95%, 98%, or 99% of sugar originally present in a mixture with one or more bioactive agents is not incorporated into carrier. In some embodiments, the amount of sugar preferentially excluded out of the carrier is about 50%.

As used herein the term “protective agent” refers one or a group of molecules that when co-incorporated with one or more bioactive agents into a carrier enhances the thermal stability, pH stability, color stability, oxidative stability, chemical stability, and/or photo stability of the one or more bioactive agents as compared to unincorporated bioactive agents and incorporated bioactive agents without one or more protective agents.

In some embodiments, one or more protective agents enhances the oxidative stability of one or more bioactive agents as compared to unincorporated bioactive agents and incorporated bioactive agents without one or more protective agents. Non-limiting examples of such protective agents include anti-oxidants, vitamin C, vitamin A, vitamin E, glutathione, uric acid, melatonin, thioredoxin, propyl gallate (PG, E310), tertiary butylhydroquinone (TBHQ), butylated hydroxyanisole (BHA, E320) butylated hydroxytoluene (BHT, E321), and EDTA, EGTA, plant derived antioxidants.

In some embodiments, plant-derived antioxidants can be rosemary, cinnamon, cassia, thyme, basil, oregano, clove, lemongrass, lavender, tarragon, lemon balm, dill, parsley, cilantro, sage, marjoram, bay laurel, dandelion, mugwort, ginseng, cardamom, black pepper, burdock, mustard, savory, nutmeg, chicory, caraway, chervil, fenugreek, pepper, celery, epazote, horseradish, wasabi, ginger, rue, sorrel, goldenrod, valerian, horehound, hibiscus, echinacea, walnut, juniper, artichoke, arugula, cardoon, holy basil, milk thistle, allspice, anise, licorice, aloe, garlic, black cohosh, witch hazel, saw palmetto, pandan, paprika, watercress, perilla, orris, vanilla, kawakawa, mint, peppermint, spearmint, wintergreen, catnip, sumac, poppy, safflower, saffron, hops, yarrow, cinchona, quassia, turmeric, lemon myrtle, lemon verbena, citrus peel and leaf including orange, lemon, grapefruit, tangerine, kumquat, kaffir, bergamot and bitter orange, pomegranate peel, olive extracts, or a combination of one or more of the above.

In some embodiments, one of more protective agents enhances the heat stability of one or more bioactive agents as compared to unincorporated bioactive agents and incorporated bioactive agents without said one or more protective agents. Non-limiting examples of such protective agents include heat shock proteins and amino acids such as arginine, proline, histidine.

In some embodiments, one of more protective agents enhances the pH stability of one or more bioactive agents as compared to unincorporated bioactive agents and incorporated bioactive agents without said one or more protective agents. Non-limiting examples of such protective agents include amino acids such as arginine, proline, histidine.

In some embodiments, one of more protective agents enhances the color stability of the one or more bioactive agents as compared to unincorporated bioactive agents and incorporated bioactive agents without said one or more protective agents. Non-limiting examples of such protective agents include organic acids such as rosmarinic acid, ursolic acid, ferulic acid, vanillic acid.

In some embodiments, one or more bioactive agents are polynucleotides and one or more protective agents are nuclease inhibitor. Non-limiting examples of nuclease inhibitor include EDTA, EGTA, RNaseIn®. In some embodiments, one or more bioactive agents are polypeptides and one or more protective agents are protease inhibitor. Non-limiting examples of protease inhibitor include trypsin inhibitor.

The term “hydrophobic” with respect to a bioactive compound refers to compounds having superior solubility in non-polar organic solvents and oils as compared to water (e.g., aqueous) and polar solvents.

The term “hydrophilic” with respect to a bioactive compound refers to compounds having superior solubility in water (e.g., aqueous) and polar solvents as compared to non-polar organic solvents and oils.

As used herein, the term “ghost cell” means a dead cell in which the outline remains visible, but without other cytoplasmic structures or stainable nucleus. In one example, the ghost cell retains the extracellular membrane or wall but lack defined nuclear and cytoplasmic details. In one example, the ghost cell can be an erythrocyte after loss of its hemoglobin. In another example, ghost cell can be epithelial cell lacking the nucleus and cytoplasmic structures. In one example, ghost cell can be bacterial cell that lack the nuclear material but retain the cell wall.

As used herein, the term “cellular aggregate” refers to cluster of cells. In some embodiments, the cellular aggregate can be produced by homogenization of tissue. In some embodiments, biological tissues (from plant or animal) that can be chemically, biochemically or mechanically be treated to create particles in the size of 0.01 μm-5 mm. In some embodiments, the carrier is a cellular aggregate. In some embodiments, the diameter of the cell aggregate is in the range of about 0.01 μm to about 5 mm. In some embodiments, the diameter of the cell aggregate is about 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 7.5 μm, 10 μm, 20 μm, 50 μm, 100 μm, 500 μm, 1 mm, 2 mm, 3 mm, 4 mm, or 5 mm.

In some embodiments, the cellular aggregate may be mechanically or chemically treated to enhance the diffusion of the compounds. The mechanical treatment may be by sonication or high pressure. The chemical treatment maybe by treating with surfactant, enzyme or acid to reduce the barriers to infusion.

As used herein, the term “high concentration buffer” refers to a buffer system in which the acid and its conjugate base or a base and its conjugate acid are present at concentration of at least about 1 M. In some embodiments, the concentration can be at least about 1 M, 1.25M, 1.5M, 1.75 M, 2 M, 2.25 M, 2.5 M, 2.75 M, 3 M, 3.25 M, 3.5 M, 3.75 M, 4 M, 4.25 M, 4.5 M, 4.75 M, or 5 M. Non-limiting examples of such buffers include citric acid-sodium citrate, acetic acid-sodium acetate, citric acid-disodium hydrogen phosphate at one of the above-mentioned concentration. In some embodiments, the pH of high concentration buffer can be about: pH 2, pH 3, pH 4, pH 5, pH 6, pH 7, or pH 8.

As used herein, the term “low concentration buffer” refers to a buffer system in which the acid and its conjugate base or a base and its conjugate acid are present at concentration of at least about 0.001 M and less than 1 M. In some embodiments, the concentration can be at least about 0.001 M, 0.00125 M, 0.0015 M, 0.00175 M, 0.002 M, 0.00225 M, 0.0025 M, 0.00275 M, 0.003 M, 0.00325 M, 0.0035 M, 0.00375 M, 0.004 M, 0.00425 M, 0.0045 M, 0.00475 M, 0.005 M, 0.01 M, 0.02 M, 0.025 M, 0.03 M, 0.035 M, 0.04 M, 0.045 M, 0.05 M, 0.055 M, 0.06 M, 0.065 M, 0.07 M, 0.075 M, 0.08 M, 0.085 M, 0.09 M, 0.095 M, 0.1 M, 0.125 M, 0.15 M, 0.175 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1 M. Non-limiting examples of such buffers include citric acid-sodium citrate, acetic acid-sodium acetate, citric acid-disodium hydrogen phosphate at one of the above-mentioned concentrations. In some embodiments, the pH of high concentration buffer can be about: pH 2, pH 3, pH 4, pH 5, pH 6, pH 7, or pH 8.

As used herein, the term “polynucleotide” refers to nucleic acids and fragments or portions thereof, which may be single or double stranded, or partially double stranded and represent the sense or antisense strand. A nucleic acid may include DNA or RNA, and may be of natural or synthetic origin and may contain deoxyribonucleotides, ribonucleotides, or nucleotide analogs in any combination. Nucleic acid may comprise a detectable label. Although a sequence of the nucleic acids may be shown in the form of DNA, a person of ordinary skill in the art recognizes that the corresponding RNA sequence will have a similar sequence with the thymine being replaced by uracil i.e. “T” with “U”.

As used herein, the term “polypeptide” refers to at least two or more amino acids linked to each other by peptide bond. In some embodiments, the polypeptide comprises about 2, 3, 4, 5, 6, 7, 8, 9, 10, 15, 20, 30, 40, 50, 60, 70, 80, 90, 100 or more amino acids linked to adjacent amino acids by peptide bond. In some embodiments, the polypeptide is a peptide hormone. In some embodiments, the polypeptide has therapeutic properties. In some embodiments, the polypeptide is an antibody.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

The phrases “loading efficiency” or “incorporation efficiency” interchangeably refer to the incorporation efficiency on both wet basis determined as follows: IE(%)=C_{E}/C_{T}×100, where C_{E} is the mass of extracted bioactive from the lipid membrane microcapsules after incorporation on a wet basis and C_{T} is the amount of bioactive initially added to the lipid membrane microcapsules.

The term “substantially all” as used herein means at least about 60%, about 70%, about 80%, about 90%, about 95%, about 99%, or 100%.

As used herein, the term “including” has the same meaning as the term comprising.

As used herein, the term “about” means in quantitative terms, plus or minus 10%.

As used herein, the term “photostability” in the context of one or bioactive agents means the stability of one or more bioactive agents in the ultraviolet wavelength range of about 100 nm-400 nm, visible wavelength range of about 390 nm to about 700 nm, infrared wavelength range of about 700 nm to about 1 mm.

As used herein the term “phytochemical” refers to naturally occurring chemicals found in plant. In some embodiments, phytochemicals comprise antioxidants. In some embodiments, phytochemicals comprise a polyphenol, carotenoid, curcuminoid, isothiocyanate, terpenoid, lignans, phenolic acids, phytosterols, fiber, chlorophyll, or a combination thereof. Non-limiting examples of polyphenols include anthocyanin, flavone, flavanone, isoflavone, flavonol, proanthocyanidin, catechin, epicatechin, procyanidin, prodelphinidin, or a combination thereof. In some embodiments, phytochemicals provide color, odor and taste. In some embodiments, phytochemicals are naturally present as a mixture with sugars, e.g., mono-, di-, or polysaccharides.

As used herein, the term “prebiotics” means food ingredient that promotes the growth of beneficial microorganisms in the intestines.

As used herein, the term “probiotics” means microorganisms that provide health benefits when consumed.

### Example 1

### Incorporation of Cranberry Extract in Lactobacillus casei

Incorporation of Cranberry Extract in Carrier

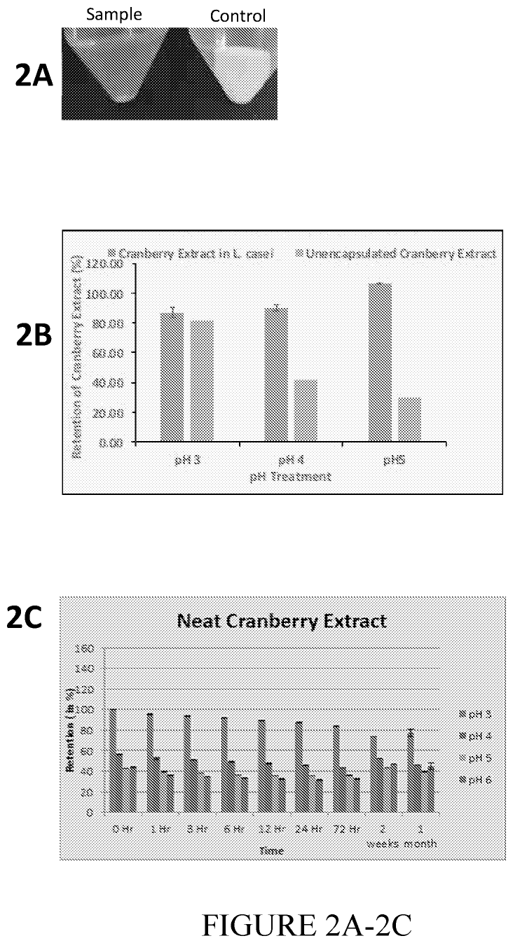

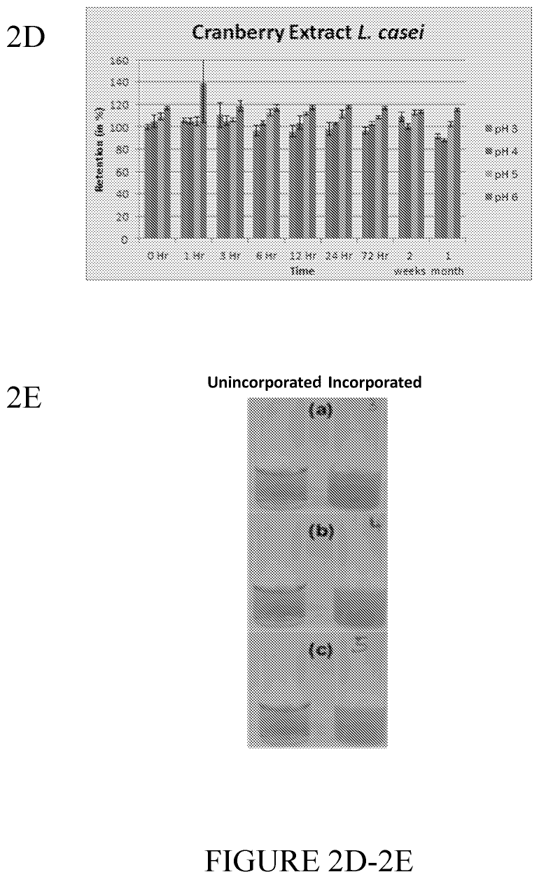

Two tubes, each containing 0.4 gram of L. casei was used, one each as control and sample. To the control tube, high concentration citric acid-sodium citrate buffer was added. To the sample tube, a solution of cranberry extract in a high concentration citric acid-sodium citrate buffer was added. Both tubes were lightly closed with a tape and transferred into vacuum bags. They were subjected to vacuum conditions. After vacuum application, the bags were incubated in the dark. The tubes were removed from the bags and centrifuged to remove unincorporated cranberry extract. Once the supernatants were discarded, low concentration citric acid-sodium citrate buffer was added to wash off the unbound cranberry extract from the cells. The cells were vortexed and centrifuged between washes. A final centrifugation was used to dry the sample and control. FIG. 2A shows the incorporated cranberry extract in L. casei cells (sample) and L. casei cells without cranberry extract.

Heat Stability Test

0.1 gram each of the incorporated sample and the control were weighed and 1 mL of citric acid-sodium citrate buffer (100 mM, pH 3) was added to each of the tubes.

Before heating—Absorbance was measured at 524 nm for the sample (non-heated), using the control as a blank.

For heating test—1 mL of the control and 1 mL of the incorporated sample were each transferred into two similar diameter thin glass tubes. The temperature of the circulating water bath was kept at 95° C. The tube was then immersed in the water. A thermocouple wire was inserted into the liquid contents of the tube. It took approximately 17 secs to reach 95° C. Once at 95° C., the timer was started and exactly after 1 min, the tube was removed and placed on ice for 1 min. After this, the contents of the control and sample tube were each transferred into disposable cuvettes. And the absorbance was measured at 524 nm.

Absorbance recorded for formulation with cranberry extract incorporated in L. casei at 524 nm.

The percent retention of the cranberry extract formulation at every pH value was calculated with respect to the absorbance measured at pH 3 before heating. FIG. 2B shows the result of a comparison of heat stability of the incorporated with the unincorporated cranberry extracts at pH 3, 4 and 5. The incorporated form shows significantly better color retention from pH 3-pH 5. For the unincorporated extract, there is color retention only at pH 3, whereas color drops significantly at pH>3.

pH Stability

L. casei with cranberry extract incorporated in it and control L. casei (0.35 g) were put into two separate 50 mL tubes. The carrier was re-suspended in 500 uL of buffer at different pH (3, 4, 5, 6). The absorbance at 524 nm was measured at time points 0, 1, 3, 6, 12, 24, 72 hours, 2 weeks and 1 month. Absorbance measurements were also taken in different pH buffers after suspending the sample carrier in the respective buffer for a one-month period. The percent retention is calculated by dividing the absorbance value at each time point with the absorbance value at the initial t=0 and pH 3. The percent color retention of un-incorporated and carrier-incorporated cranberry extract, respectively, over different time points at different pHs 3, 4, 5 and 6 are shown in FIGS. 2C and 2D. FIG. 2E shows diluted samples of the unincorporated cranberry extract and the incorporated cranberry extract in the carrier (right), suspended in buffers of pH 3, 4, 5 and 6. These were stored at ambient conditions in the respective pH buffers for nearly two months. Clearly, the unincorporated extract shows browning, but the incorporated extract in cells has retained the actual color at pH 3, 4 and 5.

Thus, excellent pH and thermal stability of cranberry extract is observed from pH 3-pH 5 when incorporated in L. casei. Additionally, superior color retention of the extract upon incorporation and storage of the extract at ambient conditions for an extended period of time (˜2 months) was observed. In conclusion, high concentration buffer for incorporation and a low concentration buffer to wash off the unbound extracts using vacuum pressure is vital for color retention and thermal stability of the extract.

### Example 2

### Incorporation of Quercetin in Saccharomyces cerevisiae and L. casei

Quercetin is a plant-based flavonoid with anti-inflammatory and immune-boosting properties. It is naturally occurring in apples and onions. Quercetin is unstable to changes in pH and oxidation. Experiments were carried out to develop incorporation systems for quercetin with microbial (yeast and Lactobacillus casei) carriers and test the systems for oxidative and pH stability.

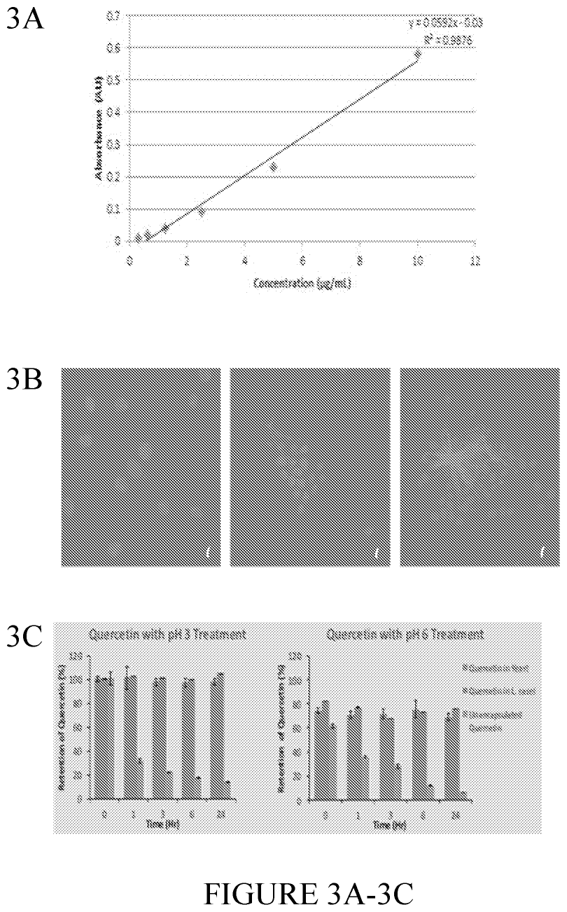

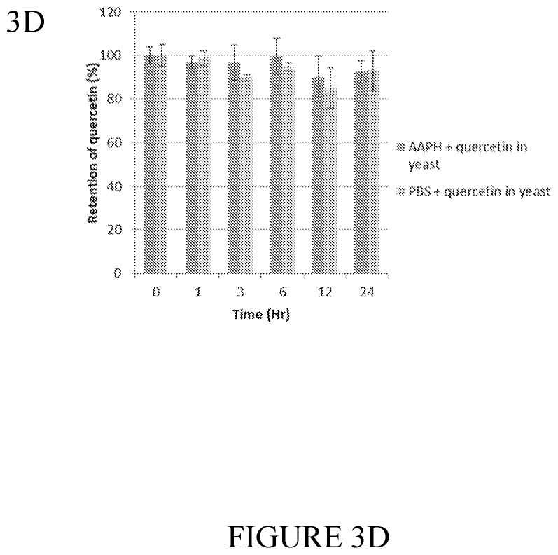

A standard calibration curve was spectrophotometrically obtained for quercetin (QU995 is the food-grade form of quercetin) in dimethyl sulfoxide (DMSO). Absorbance at 388 nm for varying concentration of quercetin in DMSO and shown in FIG. 3A.

Incorporation of Quercetin:

Incorporation of quercetin in yeast cells with ethanol-buffer mixture was achieved by a non-thermal, vacuum infusion method. Inactivated food-grade yeast cells were used to incorporate quercetin with an initial aim of achieving an incorporation yield or loading percent of 0.5%. The dry yeast cells were first washed with deionized water and an ethanol solution. The washed cells were weighed for use as control (without quercetin) and sample (with quercetin). The incorporation was done in high vacuum conditions and the control and sample were incubated in the dark. The unincorporated components were removed by centrifugation and by discarding the supernatants. Water and ethanolic washes of the control and sample were done to remove any remaining unincorporated or unbound material.

Extraction:

A total of 10 mg each of quercetin incorporated in yeast (sample) and yeast without quercetin (control) were weighed out. 1 mL DMSO were added to both tubes and sonicated. The sample and control were centrifuged. A 500 μL of supernatant was removed and measured absorbance at 388 nm. The yeast control was used as the blank. The incorporation yield or loading percent is the amount of bioactive loaded per mass of yeast cells. Subsequent extraction of quercetin using dimethyl sulfoxide gave an incorporation efficiency of 65% and an incorporation yield (or loading %) of 0.35% (on a wet basis).

Choice of Buffer:

Quercetin incorporation was performed in different buffers to determine the optimum buffer conditions for efficient incorporation. The buffers tested were-low concentration citric acid-sodium citrate buffer, and low concentration sodium phosphate buffer. Multiphoton imaging (FIG. 3B) of quercetin incorporated with the phosphate buffer showed clear infusion of quercetin into yeast cells, without any crystal formation on top of or near the cells (FIG. 3B(a)). On the other hand, when the incorporation was done in citric acid-sodium citrate buffer, some clumping of tiny quercetin crystals was observed (FIG. 3B(b)), and with citric acid-sodium citrate buffer, pH 6, there were a large amount of tiny crystals (FIG. 3B(c)). Thus, phosphate buffer proved more desirable for the incorporation of quercetin.

pH Stability for Quercetin in Yeast, in L. casei and Unincorporated Quercetin

A comparative pH test at different pH's (3, 4, 5, 6) was done with quercetin incorporated in yeast, in L. casei and un-incorporated quercetin. At every time point, an extraction was done to spectrophotometrically determine the amount of quercetin retained. Quercetin incorporated in yeast and in L. casei is much more stable over the range of pH. In contrast, unincorporated quercetin (gray) shows rapid degradation over time (FIG. 3C). The percent retention is calculated by dividing the absorbance value at each time point with the absorbance value at the initial t=0.

Oxidative Stability Data for Quercetin in Yeast (Single Infusion)

Quercetin has a tendency to undergo oxidation by air, especially in water under ambient conditions. The effect of oxidation on the incorporated quercetin was studied using a free radical initiator AAPH (2,2′-azobis (2-methylpropionamidine dihydrochloride). Incorporated quercetin in yeast treated with AAPH was compared with quercetin incorporated in yeast suspended in phosphate buffer saline (PBS) buffer. FIG. 3D represents the amount of quercetin retained with and without the initiator over a 24-h period. The results indicate that the yeast incorporated quercetin is well protected from oxidative degradation even in the presence of strong oxidation initiators over a long period of time.

Multiple Infusions of Quercetin in Yeast and L. casei

Methods to improve the incorporation yields of quercetin in yeast and L. casei were explored. One such method is multiple infusions of the bioactive into the microbial carrier cells. A similar protocol as detailed above was used to incorporate quercetin in yeast and L. casei. For double infusion, the same amounts of quercetin, buffer and ethanol were added to the cells infused for the first time with quercetin and the unincorporated components were similarly removed by wash and centrifugation steps. For a quadruple infusion, the cells were treated the same way after infusing three times with quercetin. The problem encountered in a quadruple infusion was crystallization of quercetin around carriers, instead of being incorporated (determined by a multiphoton fluorescence imaging).

A double infusion of quercetin in the second carrier was carried out similar to the method adopted for the double infusion of quercetin in yeast. A 1.5-fold increase in incorporation yield was observed upon a double infusion (Table 1)

TABLE 1
Incorporation yields and efficiencies
for quercetin in L. casei and yeast

| | Buffer used for | | |
|---|---|---|---|
| | incorporation with | Incorporation | Incorporation |
| | 35% ethanol | Yield (%) | Efficiency (%) |
| Quercetin in | | | |
| L. casei | | | |
| Single | Citric acid sodium | 0.46 | 80.05 |
| infusion | citrate | | |
| | (0.1M, pH 3) | | |
| Single | Sodium phosphate | 0.83 | 145.25 |
| infusion | (0.1M, pH 5.8) | | |
| Double | Sodium phosphate | 1.29 | 112.88 |
| infusion | (0.1M, pH 5.8) | | |
| Quercetin | | | |
| in Yeast | | | |
| Single | Sodium phosphate | 0.56 | 98.44 |
| infusion | (0.1M, pH 5.8) | | |
| Double | Sodium phosphate | 1.26 | 109.52 |
| infusion | (0.1M, pH 5.8) | | |

Comparison with Conventional Industrial Methods:

Oil and water emulsions are an alternative method for encapsulating hydrophobic compounds such as quercetin. Not only was the process for encapsulating quercetin in an oil and water emulsion time consuming, emulsion also do not have the same loading capacity for encapsulating quercetin as our microbial carriers. Incorporation efficiency was only ˜15%.

Improved Quercetin Delivery:

Based on the incorporation experiments, inventors discovered that on a gram to gram basis, cell incorporation process can deliver 545 times more quercetin than the average apple. The amount of quercetin in an average apple is 0.044 mg/g. In comparison, the amount of quercetin that can efficiently incorporate into the cells is 12.517 mg/g.

Accordingly, quercetin can be successfully incorporated in yeast and L. casei. The pH and oxidative stability of incorporated quercetin improved (5-8 fold) in comparison to the unincorporated form. The incorporation yield or the loading percent sufficiently improved by employing a double infusion strategy.

### Example 3

### Incorporation of Purple Carrot Extract in Several Microbial Organisms

Purple carrot is a vegetable rich in anthocyanins which impart it the characteristic deep color. As the pH of the solution changes from acidic to basic, the anthocyanins undergo a reversible change in molecular structure. This structural change is characterized by a shift from red to purple to blue with increasing pH. The purple carrot extract is used as a natural food colorant. But changes in food processing and storage that are associated with changes in temperature and pH make this natural colorant extract unstable, changing color or fading in color.

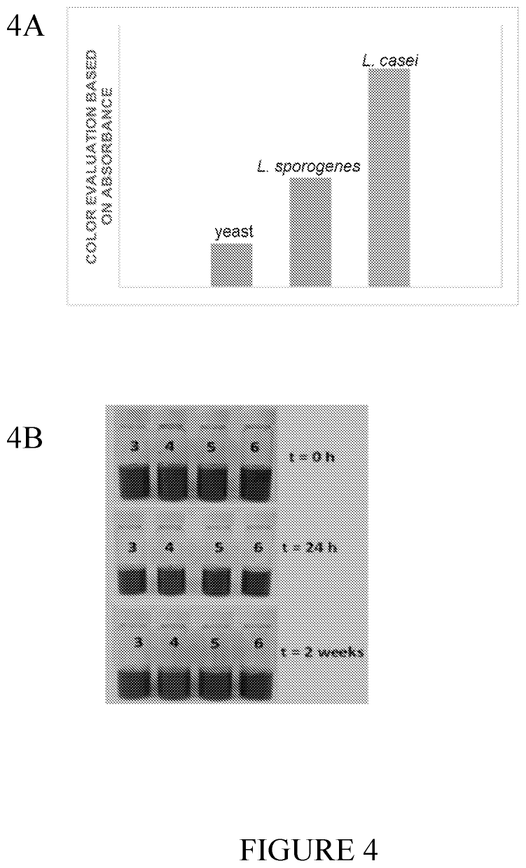

Incorporation systems for purple carrot extract with microbial carriers were developed and the systems were tested for color (pH) and thermal stability. Initial incorporation testing with yeast, L. sporogenes and L. casei showed greater color stability with L. casei (FIG. 4A). Therefore, L. casei was employed as the carrier for water-soluble purple carrot extract. Comparison of stability of the incorporated with the unincorporated purple carrot extract as well as with conventional industrial incorporation methods were performed. Incorporation of purple carrot extract in L. casei: The washed L. casei cells were weighed for use as control (without purple carrot extract) and sample (with purple carrot extract). The control and sample cells were suspended in a high concentration buffer. The incorporation was done in vacuum conditions and the control and sample were incubated in the dark for 10 min. Any unincorporated or unbound extract was removed by centrifugation and by discarding the supernatants. Low concentration buffer washes of the control and sample were done to remove any remaining unincorporated or unbound material.

A small amount of the sample was weighed and suspended in low concentration citric acid-sodium citrate buffer. The control (L. casei without purple carrot extract) was treated the same way. Absorbance measurement of the suspension at 524 nm using the control as the blank gave a value that ensured the successful incorporation of purple carrot extract in L. casei.

Incorporation of Purple Carrot Extract in Yeast:

Dry yeast (1 g) was weighed into 50 mL centrifuge tube. The yeast was washed two times with ethanol solution and one time with water. The yeast cell suspension was centrifuged between washes. One final centrifugation was done to remove excess water. Washed yeast (1 g) was weighed into two 50 mL tubes. To one tube, high concentration citric acid-sodium citrate buffer was added to the control. To another tube high concentration citric acid-sodium citrate buffer was added to the sample followed by adding purple carrot extract. The sample and control were subjected to vacuum conditions. The tubes were incubated in the dark. The samples were removed from vacuum bags and centrifuged. The sample was washed with low concentration citric acid-sodium citrate buffer. The sample was centrifuged between washes. One final “drying” centrifugation was done to remove excess buffer.

Purple Carrot Extract Incorporation in L. sporogenes

Lactospore powder (5 g) was weighed two times into 50 mL tubes and washed with milliQ water. The L. sporogenes preparations were washed two times with ethanol solution and one time with water. The L. sporogenes was centrifuged between washes. One final “drying” spin was done to remove excess water. Washed L. sporogenes (1 g) was weighed into two separate 50 mL tubes. For control high concentration citric acid-sodium citrate buffer was added. For sample, high concentration citric acid-sodium citrate buff was added, followed by adding purple carrot extract. The sample and control were subjected to vacuum conditions. The tubes were incubated in the dark. Samples were removed from vacuum bags and centrifuged to remove un-incorporated purple carrot solution. The suspension was and washed with low concentration citric acid-sodium citrate buffer. The L. sporogenes were centrifuged between washes. One final “drying” spin was done to remove excess buffer.

Incorporation of water-soluble purple carrot extract in different microbial carriers (yeast, L. sporogenes and L. casei) has shown improved color retention in the following order—yeast (S. cerevisiae)<L. sporogenes<L. casei. This is depicted in FIG. 4A.

pH Stability:

Unincorporated purple carrot extract changes color from red to purple to blue in the pH range of 3 to 5. At pH 5 and above, the anthocyanin pigments in the extract breakdown, resulting in a significant loss of color (extremely light purple). This is also evident in very low absorbance values of the unincorporated extract solution. The color stability of purple carrot extract incorporated in L. casei at pH 3, 4, 5 and 6 were studied by suspending the L. casei incorporated extract in food-grade citric acid-sodium citrate buffer at different pH and spectrophotometrically determining (absorbance measurements) the stability over a period of one month. Adequate color retention in the pH range of 3 to 5. At pH 6, browning is observed (FIG. 4B) indicating that incorporating purple carrot extract in L. casei enhances color stability at pH 3-5 over a period of one month.

### Example 4

### Incorporation of Retinol in L. casei and Yeast (S. cerevisiae)

Retinol in anti-aging products are highly susceptible to degradation due to various factors, such as oxygen and light. Incorporation systems with microbial carriers were developed for retinol and the systems were tested for oxidative stability and improve the loading or incorporation yield in the carriers.

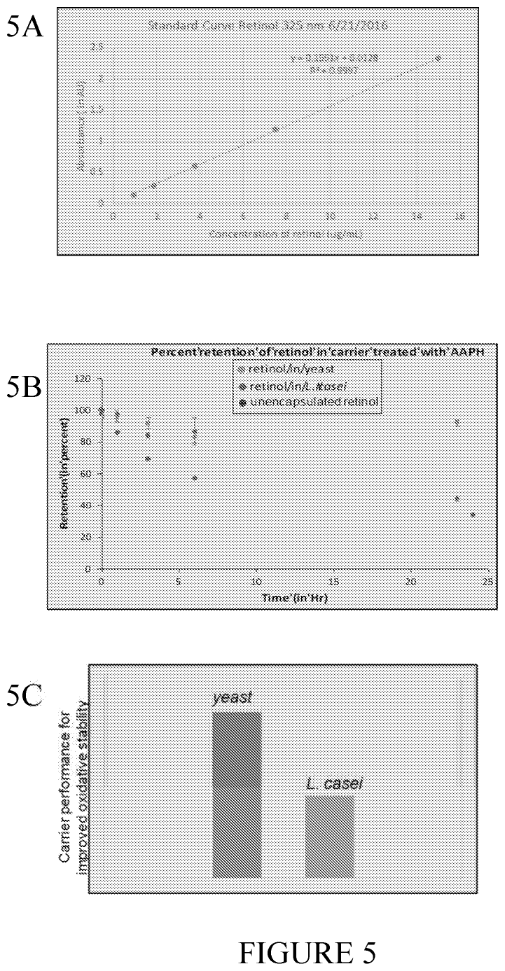

A standard calibration curve was spectrophotometrically obtained for retinol in methanol (FIG. 5A). Retinol was kept in a vacuum sealed bag to prevent any oxidation. Incorporation of retinol in L. casei as well as in yeast cells was performed with ethanol-phosphate buffer mixture by a non-thermal, vacuum infusion method. For both L. casei and yeast, the following protocol was used—The washed cells were weighed for use as control (without retinol) and sample (with retinol). The incorporation was done in vacuum conditions and the control and sample were incubated in the dark. The un-incorporated components were removed by centrifugation and by discarding the supernatants. Water and ethanolic washes of the control and sample were done to remove any remaining unincorporated or unbound material. Care was taken to preserve the incorporated samples in vacuum-sealed bags after the experiment and samples were stored at 4° C. until further use.

Improved Incorporation of Retinol in L. casei:

Incorporation of retinol was also performed multiple times with L. casei as carrier to increase the incorporation yield. Multiple rounds of incorporation in L. casei significantly improved the yield. A doubling of the percent yield was observed with every additional incorporation. In the case of yeast, multiple incorporations were not attempted as precipitation of retinol was suspected to occur during the multiple infusions. Table 2 depicts the incorporation yields obtained for retinol in L. casei and yeast.

TABLE 2
Incorporation Yields Obtained for Retinol in L. casei and yeast.

| | Number of | | | Experimentally | |
|---|---|---|---|---|---|
| | times vacuum | | | obtained | Theoretical |
| Microbial | incorporation | | Concentration | incorporation | incorporation |
| carrier | done | Absorbance | (μg/mL) | yield (%) | yield (%) |
| L. casei | 1x | 1.948 | 62.36 | 0.6236 | 0.9 |
| | | (1:5 dilution) | | | |
| L. casei | 2x | 0.679 | 95.72 | 0.9572 | 1.5 |
| | | (1:20 dilution) | | | |
| L. casei | 4x | 0.991 | 153.7 | 1.537 | 3.2 |
| | | (1:20 dilution) | | | |
| Yeast | 1x | 1.283 | 93.58 | 0.9358 | 0.9 |
| | | (1:10 dilution) | | | |

Oxidative Stability Data for Retinol in Yeast (Single Infusion) and L. casei (Quadruple Infusions):

The effect of oxidation on the yeast-incorporated retinol and L. casei-incorporated retinol were studied using a free radical initiator AAPH (2,2′-azobis(2-methylpropionamidine) dihydrochloride). In both cases, retinol incorporated in the microbial carrier subjected to AAPH exposure was compared with retinol suspended in phosphate buffer saline (PBS) buffer, without any AAPH added. FIG. 5B represents the amount of retinol retained with and without the initiator over a 24-h period in yeast and L. casei. The results indicate that the yeast incorporated retinol is significantly well protected from oxidative degradation than the L. casei incorporated retinol over a 24-h time period.

Between the two carriers, yeast is better than L. casei for enhancing oxidative stability of retinol (FIG. 5C). From our other preliminary studies, we have also discovered that yeast fares better than L. casei for improving light stability of hydrophobic molecules.

### Example 5

### Enhanced Color Stability by the Use of Protective Agents

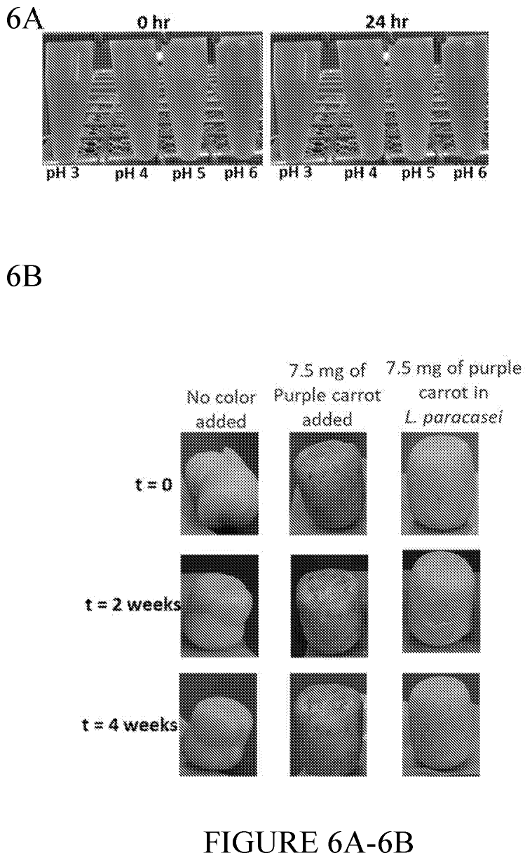

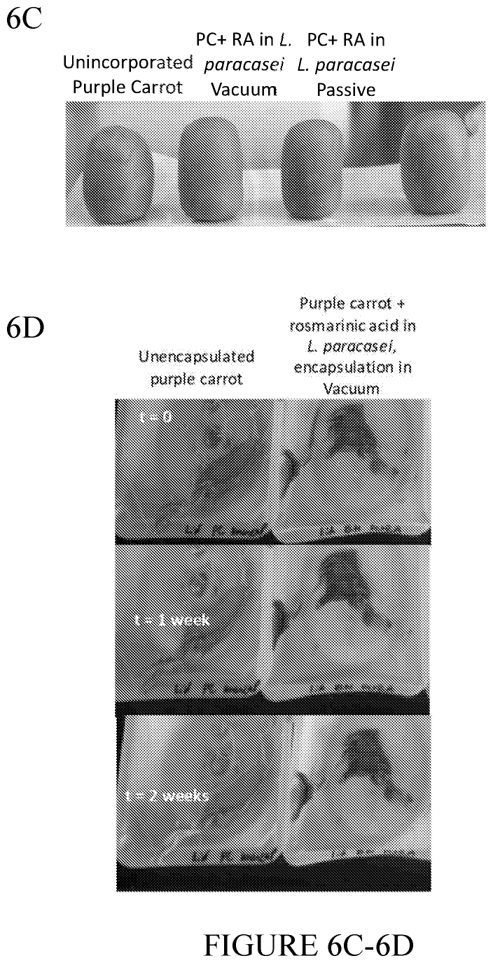

Purple carrot extract in 100 mM citric acid-sodium citrate at the different pHs were incorporated in Lactbacillus paracasei together with rosmarinic acid extract as a protective agent and evaluated for the color intensity at 0 hour and 24-hour time points. The color intensity was retained even after 24 hours (FIG. 6A) indicating that the protective effect of incorporation into L. paracasei and that of rosmarinic acid.

### Example 6

### Enhanced Color Stability by the Incorporation of Purple Carrot Extract, by Co-Incorporation of Protective Agents, and by Coating with a Biopolymer and Testing in a Commercial Product

The enhanced color protective effects of incorporation in a carrier of purple carrot extract, co-incorporation of a protective agent together with purple carrot extract in a carrier, and coating of the incorporated purple carrot extract were tested in a chewing gum product.

Different freeze dried colorant samples were tested by mixing with chewing gum. In one case, the colorant was freeze dried unincorporated purple carrot extract. In another case, the colorant was freeze dried mixture of purple carrot extract incubated overnight with L. paracasei and rosmarinic acid. In another case, purple carrot extract and rosmarinic acid were incubated with L. paracasei for 10 min, followed by co-incorporation under vacuum for 5 sec. In another case, the purple carrot extract was incubated with L. paracasei for 10 min, followed by incorporation under vacuum for 5 secs and then coated with chitosan and freeze dried.

The gum was made by heating 5 g gum base for 1 min in a microwave. Corn syrup and the freeze dried coloring agent was mixed with the gum base, and microwaved for 15 sec. The microwaved mixture was then placed on powdered sugar and mixed. The resulting colored gum products were shown in FIG. 6B.

The color intensity of the gum without colorant, gum mixed with unincorporated purple color extract and gum mixed with purple carrot extract and rosmarinic acid incorporated in L. paracasei were tested for several weeks. The gum mixed with purple carrot extract and rosmarinic acid incorporated in L. paracasei retained its pink color for at least 4 weeks as shown in FIG. 6B. Thus, incorporation of purple carrot extract enhances its color stability.

The chewing gum was prepared first and then the coloring agents: unincorporated purple carrot extract; purple carrot extract, mixed with rosmarinic acid and incorporated in L. paracasei under vacuum; purple carrot extract, mixed with rosmarinic acid and incorporated in L. paracasei by passive diffusion; purple carrot extract incorporated in L. paracasei under vacuum and the gum is finally coated with chitosan were added to the prepared gum ball. The samples were placed these samples in a 37° C. incubator to observe color loss or retention over a period of 2 weeks. The gum balls with purple carrot extract, mixed with rosmarinic acid and incorporated in L. paracasei under vacuum retained the color most as shown in FIG. 6C.

To test the color stability, gum with unincorporated purple carrot extract and gum with purple carrot extract and rosmarinic acid incorporated in L. paracasei were placed on a weigh boat. The weigh boats were placed on the surface of boiling water in a container for 2 minutes. There was a slight loss in red color of the unincorporated purple carrot in comparison to incorporated product. Upon keeping the heated gum samples at 37° C., there was clearly loss of color from 0 to 2 weeks for the unincorporated purple carrot. In contrast, good color retention was observed for the gum with incorporated purple carrot extract (FIG. 6D).

### Example 7

### Enhanced Color Stability by the Incorporation of Purple Carrot Extract with High Concentration Buffer

The ability to enhance the color stability at different buffer concentrations were tested. The chewing gum was prepared first. The purple carrot extract with rosmarinic acid incorporated in L. paracasei under vacuum and prepared in either low concentration buffer, e.g. 100 mM Citrate buffer or high concentration buffer, e.g., 2M Citrate buffer were mixed with the gum ball and incubated at 37° C.

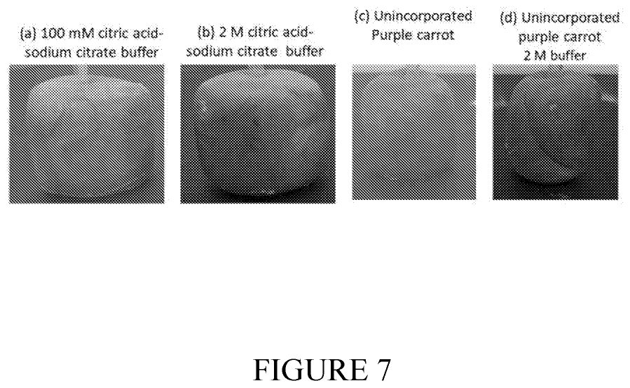

High concentration buffer, e.g. 2M Citrate buffer provided a better color protection than low concentration buffer (FIG. 7) of incorporated purple carrot extract.

### Example 8

### Estimation of Total Phenolics in Incorporated Inactivated Cells By Folin—Ciocalteau Assay

Variety of fruit juice concentrates, e.g., strawberry concentrates, cranberry concentrates, grape concentrates, raspberry concentrates, blackberry concentrates, purple carrot extracts comprising phenolics (e.g., anthocyanins, proanthocyanidins), were incorporated into carriers (e.g., microbial cells) using the methods disclosed above in this application. The goal was to concentrate as much of phenolics present in the fruit juice concentrates into the into carriers (e.g., microbial cells) and preferentially remove or decrease the amount of sugar during the incorporation process.

Estimation of Total Phenolics:

Estimation of total phenols in a solution was determined using the Folin-Ciocalteu reagent (Methods in Enzymology 1999; 299: 152-178). A 2N Folin-Ciocalteu's reagent was obtained from Sigma-Aldrich (USA). The reagent will react with phenols and non-phenolic reducing substances to form chromogens that can be detected spectrophotometrically. Gallic acid (also known as 3,4,5-trihydroxybenzoic acid) is a trihydroxybenzoic acid, a type of phenolic acid. It reacts with Folin-Ciocalteu's reagent to generate chromogens which are detected spectrophotometrically. Absorbance of each solution was determined at 765 nm. Concentration of total phenolics in a solution are expressed as gallic acid equivalents.

Calibration Curve of Gallic Acid

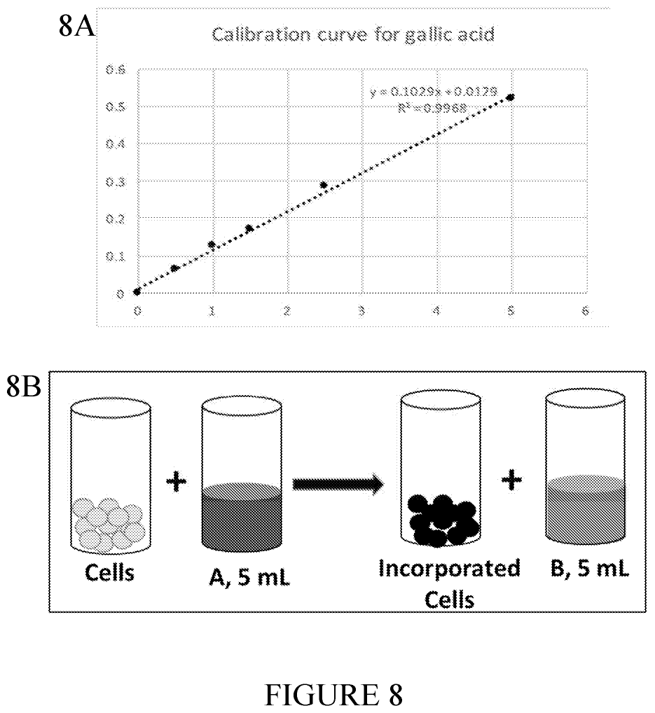

Gallic acid stock solution was prepared by dissolving 0.5 g of dry gallic acid in 10 ml of ethanol and the volume was diluted with distilled water to 100 ml. To 100 mL volumetric flasks, 0, 1, 2, 3, 5 and 10 mL of the above gallic acid stock solution was added and then diluted to 100 ml with distilled water. The above solutions have phenol concentrations of 0, 50, 100, 150, 250 and 500 mg/L gallic acid. From each calibration solution, sample, or blank, 20 uL was pipetted onto a cuvette, and to each 1.58 mL water was added, mixed and 100 uL of the Folin-Ciocalteau reagent and mixed well. The solution was incubated for ˜5 min., and then 300 uL of the sodium carbonate solution was added and shaken to mix. The solutions were kept in a 40° C. incubator for 30 min. Absorbance of each solution was determined at 765 nm against the blank (“0” mL solution) and plotted absorbance versus concentration. The absorbances for the corresponding concentrations of gallic acid are shown in the Table 3 below and the calibration curve generated is shown in FIG. 8A.

TABLE 3
Absorbance of gallic acid at various concentrations

| starting | diluted | | |
|---|---|---|---|
| standards | standards | | |
| concentration | conc for assay | | |
| (mg/L) | (mg/L) | AVERAGE | STDEV |
| 0 | 0 | 0 | 0 |
| 50 | 0.5 | 0.06166667 | 0.0085049 |
| 100 | 1 | 0.12466667 | 0.00929157 |
| 150 | 1.5 | 0.16866667 | 0.00814453 |
| 250 | 2.5 | 0.285 | 0.01442221 |
| 500 | 5 | 0.51833333 | 0.00750555 |

FIG. 8B shows an exemplary schematic of the incorporation of phenolics into carriers (e.g., microbial cells) after addition of fruit/vegetable concentrate into carriers (e.g., microbial cells) and the formation of incorporated carriers (e.g., microbial cells) with the supernatant containing the unincorporated or unbound phenolics. Briefly, the concentration of the total phenolics were estimated of the starting material (e.g., fruit juice concentrate) prior to incorporation into carriers (e.g., microbial cells). Following the incorporation process, the carriers comprising phenolics were separated and the concentration of the total phenolics in the remaining material is again estimated. The concentration of the phenolics incorporated into the carriers (e.g., microbial cells) are determined by the difference of the concentration of the total phenolics in the starting material (e.g., fruit juice concentrate) prior to incorporation (FIG. 8B A) and the concentration of total phenolics in the remaining material (FIG. 8B B). The concentrations of the total phenolics are expressed as gallic acid equivalents.

Microbial Cells:

L. paracasei was obtained from Probi Inc., Seattle, Wash. and Torula yeast (Candida utilis) was obtained from Ohly, Wis. Commercially obtained L. paracasei is normally standardized with corn maltodextrin. Therefore, these bacterial cells were washed thoroughly with distilled water to remove the corn maltodextrin. To 1 g of dry L. paracasei cells, 10 mL of distilled water was added. The cell contents were vortexed, centrifuged at 5000 rpm for 5 min and the supernatant discarded. This water wash procedure was repeated a second time to completely remove all of the corn maltodextrin.

Incorporation Process:

Buffer used in the incorporation process is 10 mM citric acid-sodium citrate (CA-SC) at pH 3. To the washed cell slurry, 1 mL fruit juice concentrates comprising phenolics (e.g., anthocyanins, proanthocyanidins)+4 mL corresponding 10 mM CA-SC buffer, pH 3 was added. This was vortexed well and left to incubate overnight at room temperature by mixing on a rotating device. The next day, the contents were centrifuged at 5000 rpm for 5 min and the supernatant was preserved for the assay. To the cell residue, 5 mL of distilled water was added, vortexed and centrifuged at 5000 rpm for 5 min. The water washes were done another three times to ensure removal of all of the unincorporated berry concentrate.

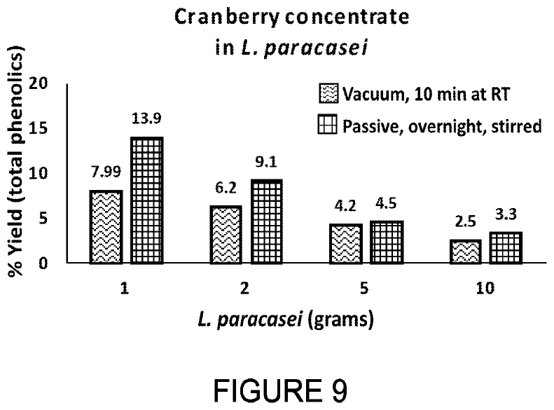

In one example, the goal was to determine if the more of the total phenolics could be incorporated upon increasing cell mass. Accordingly, the same protocol was repeated for different cell masses (2 g, 5 g and 10 g), keeping the amount of berry concentrate same (1 mL cranberry concentrate+4 mL of 10 mM CA-SC buffer, pH 3). FIG. 9 shows % incorporation yields of total phenolics from cranberry concentrate in L. paracasei. The % Yield indicates the amount of phenolics in cells/original mass of cells taken for incorporation. The results indicate a 1:1 ratio of cell:berry concentrate works best for achieving a high concentration of phenolics in cells

### Example 9

### Incorporation of Various Fruit and Vegetable Concentrates into Various Carriers AND Estimation of Total Phenolics Incorporated

The above protocol described in Example 8 for incorporation was employed for incorporation of various fruit and vegetable juice concentrates (cranberry, strawberry, raspberry, blackberry, grape and purple carrot) into various carriers. In all these cases, 1 g of cells were used for the incorporation. In the case of L. paracasei, upon washing off the corn maltodextrin, only ⅕th of the cells remained. For example, if 1 g of cells were taken to begin with, water washes to remove the corn maltodextrin left 0.2 g of the wet cell slurry. Drying this wet slurry yields 1/30th of the original mass of powder taken. In the case of torula yeast (Candida utilis), if 1 g of dry cells were taken, water washes doubled the amount of torula yeast, making the quantity 2 g of wet cell slurry. But drying yields 0.2 g of dry yeast powder. Four different incorporations were run for each fruit and vegetable concentrate—1) 10 mM CA-SC buffer for incorporation, water washes; 2) 100 mM CA-SC buffer for incorporation, water washes; 3) 2 M CA-SC buffer for incorporation, water washes; 4) 2 M CA-SC buffer for incorporation, 100 mM CA-SC buffer washes. The amount of total phenolics incorporated were determined by methods described above.

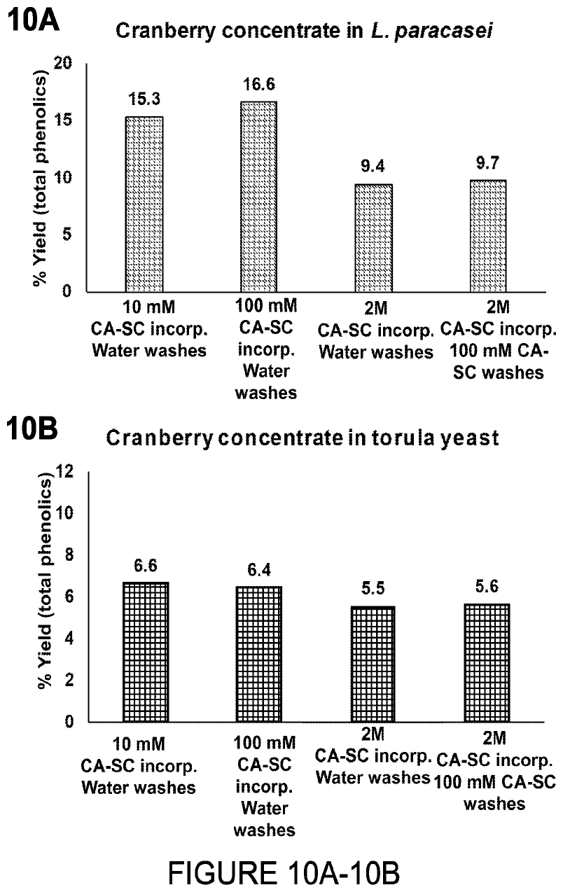

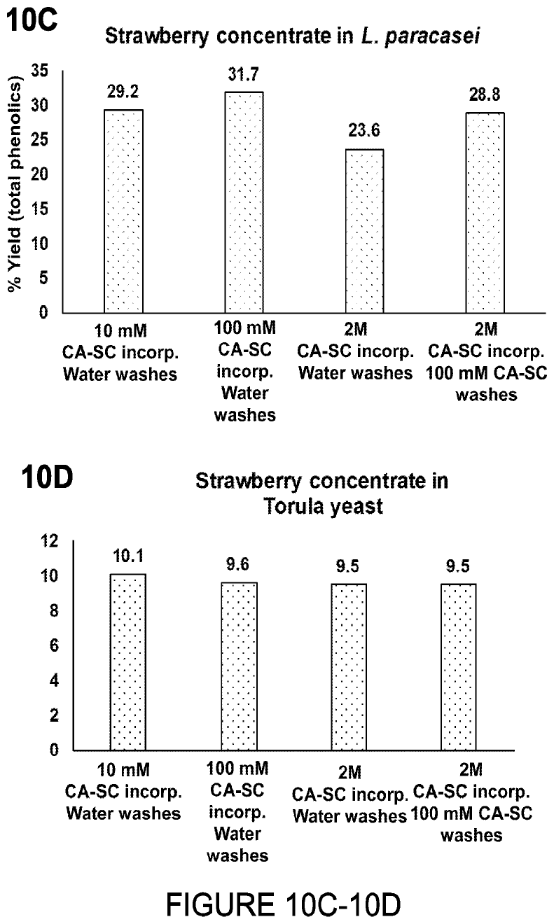

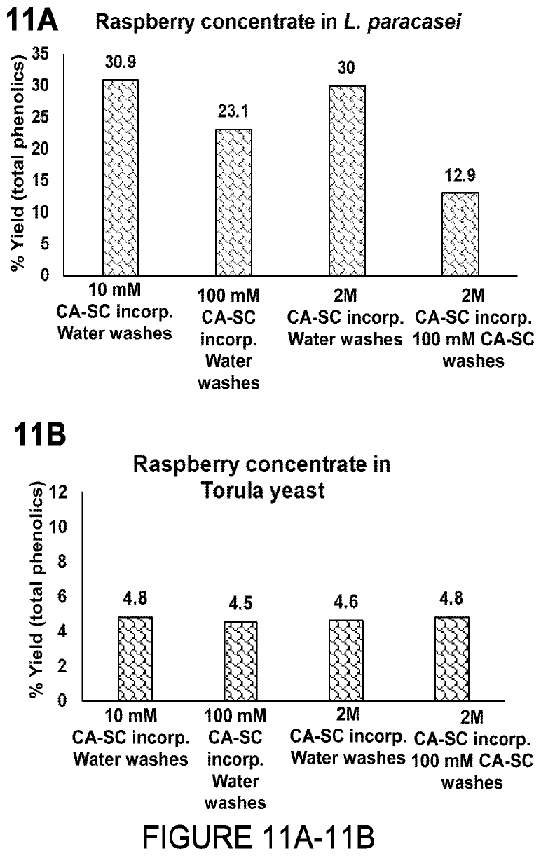

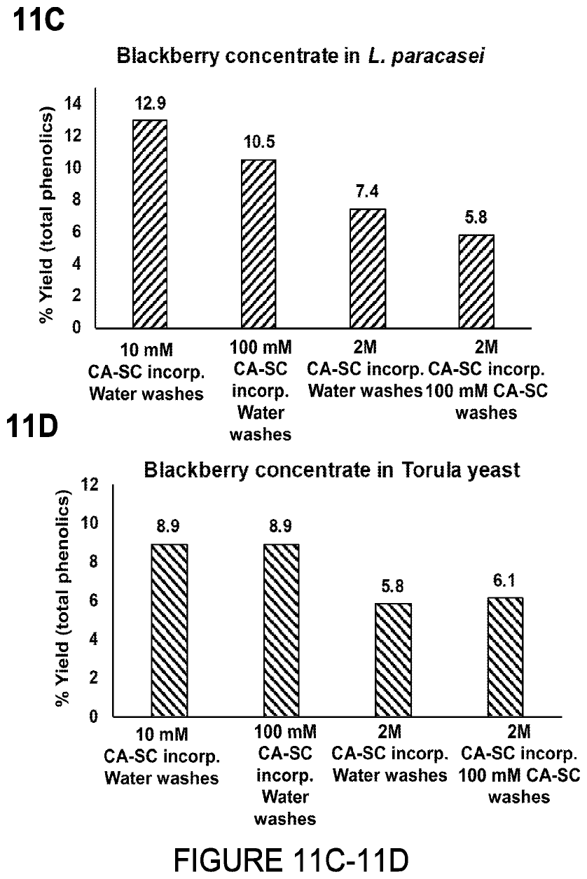

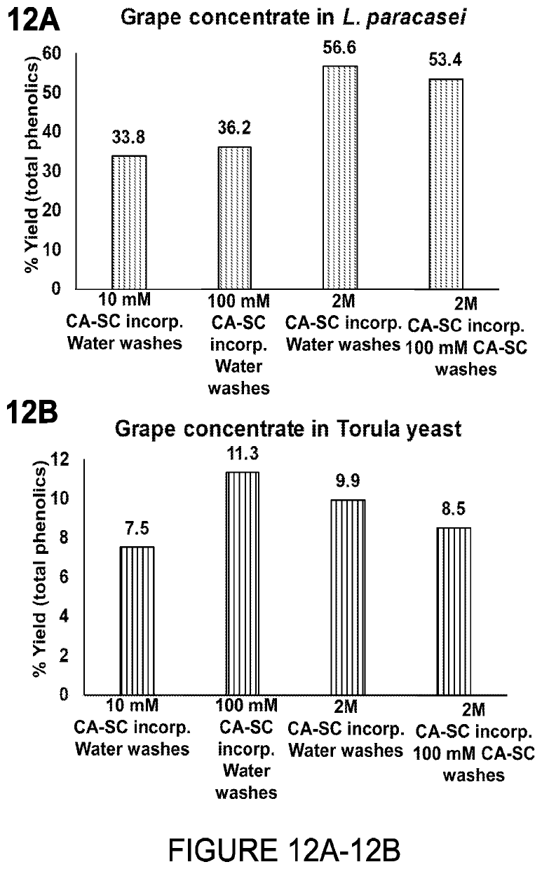

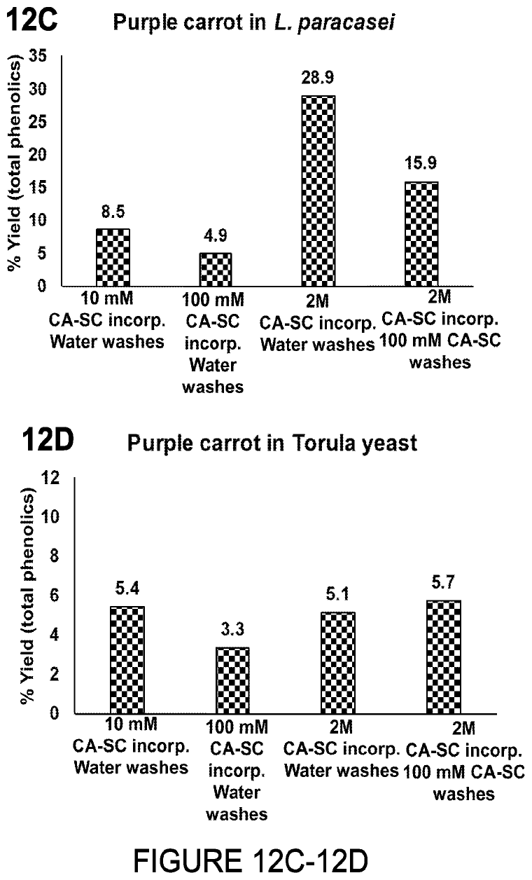

A significant amount of phenolics (antioxidants) are incorporated in the cells as is evident from the % incorporation yields. While a minor trend in % yield with changes in buffers used for incorporation and washes with the cranberry formulations was observed (FIG. 10 A), this trend was not observed for other fruit or vegetable juice concentrates (FIGS. 10C and 10D for strawberry, FIGS. 11A and B for raspberry and FIGS. 11 C and D for blackberry and FIGS. 12 A and B for grape and FIGS. 12 C and D for purple carrot formulations).

### Example 10

### Preferential Exclusion of Sugar from Different Fruit and Vegetable Concentrates During Incorporation into Various Carriers

To determine if sugars (e.g., sucrose, fructose, glucose, etc.) were preferentially removed from the carriers (e.g., L. paracasei or Torula yeast) during the incorporation process of different fruit and vegetable extracts, Brix measurements were performed.

Degrees Brix is the sugar content of an aqueous solution. One-degree Brix is 1 gram of sucrose in 100 grams of solution and represents the strength of the solution as percentage by mass. Degrees Brix is usually used in the fruit industry for fruit juices, drinks and other sugar-rich products. These products contain mostly various sugars, particularly glucose, fructose and sucrose in addition to water. However, various acids, such as citric or malic acid, as well as polymeric sugars also affect density and the refractive index. A liquid has one-degree Brix (=1% Brix) if it has the same refractive index (n) as a solution of 1 g sucrose in 100 g of water. It has 10 Brix (=10% Brix) if the n is that of a solution of 10 g sucrose in 100 g of sucrose water solution (equal to a ten percent solution).

A Brix refractometer was used to perform the measurement. The amount of sugars present in the starting solution (solution of the fruit concentrate+buffer) used for incorporation and the amount present in washes (supernatant and water washes) after incorporation were measured using the Brix refractometer. All measurements were done in triplicate.

The starting solution refers to what is added to the cells prior to incorporation. This is equal to 1 mL of the fruit concentrate+4 mL of buffer, which gives a Brix value of 10 degree for cranberry and ˜15 degree for the other fruit concentrates. The sum of the Brix values for the supernatant and washes is equivalent to that of the starting solution. These results indicate that the sugars are not taken in by the cells.

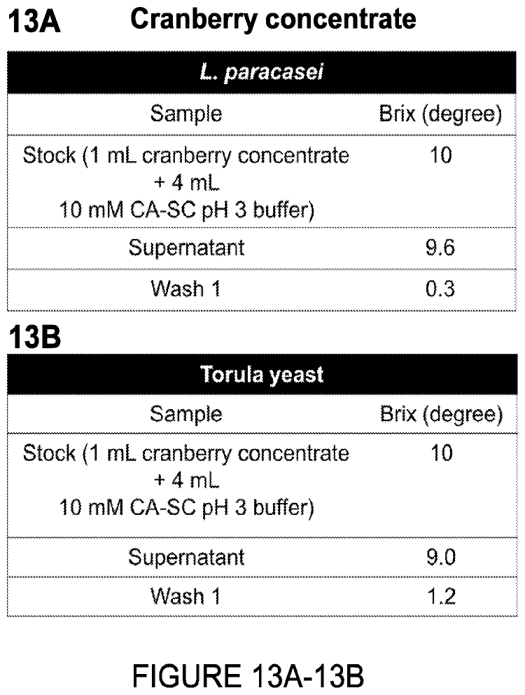

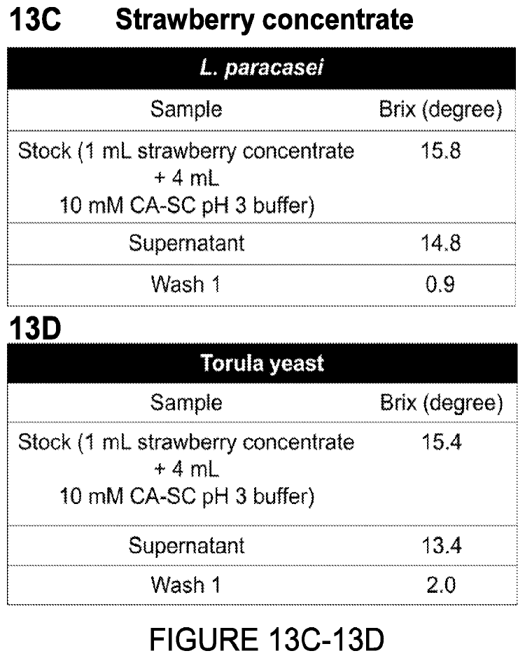

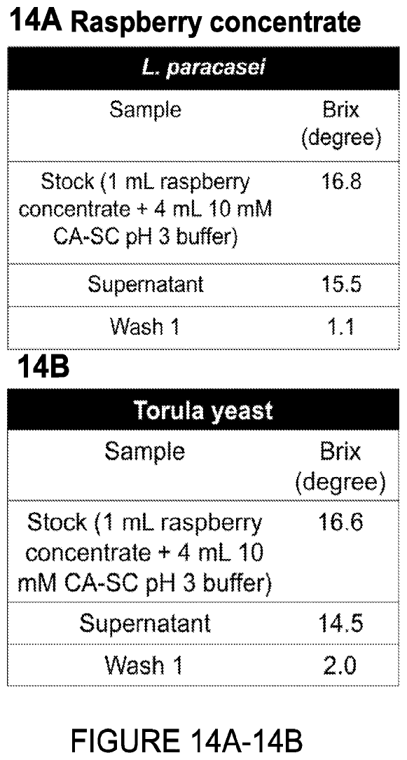

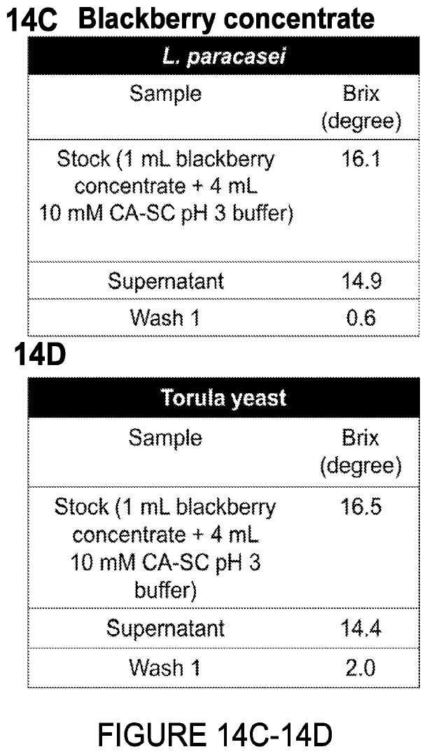

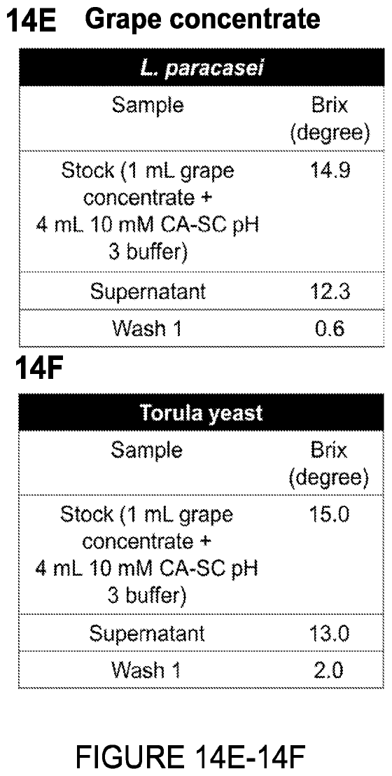

Inventors of the present application have devised a method wherein the cells only preferentially incorporate the phenolics but leave out the sugars. The results of preferential exclusion of sugars during incorporation of Cranberry concentrate by L. paracasei and Torula yeast are shown in the tables in FIGS. 13A and 13B, respectively. The results of preferential exclusion of sugars during incorporation of Strawberry concentrate by L. paracasei and Torula yeast are shown in the tables in FIGS. 13C and 13D, respectively. The results of preferential exclusion of sugars during incorporation of Raspberry concentrate by L. paracasei and Torula yeast are shown in the tables in FIGS. 14A and 14B, respectively. The results of preferential exclusion of sugars during incorporation of Blackberry concentrate by L. paracasei and Torula yeast are shown in the tables in FIGS. 14C and 14D, respectively. The results of preferential exclusion of sugars during incorporation of Grape concentrate by L. paracasei and Torula yeast are shown in the tables in FIGS. 14E and 14F, respectively.

These results indicate that the sugars are preferentially excluded by the carriers (e.g., cells). The carriers (e.g., cells) preferentially incorporate the phenolics but leave out the sugars.

### Example 11

### Masking of Taste of Incorporated Fruit and Vegetable Extract

Taste masking of bitter or astringent flavors in food/beverages in important for wider consumer experience. In this regard, cranberry extract was chosen in the formulations as cranberry has a very strong sourness/bitterness attributed to it.

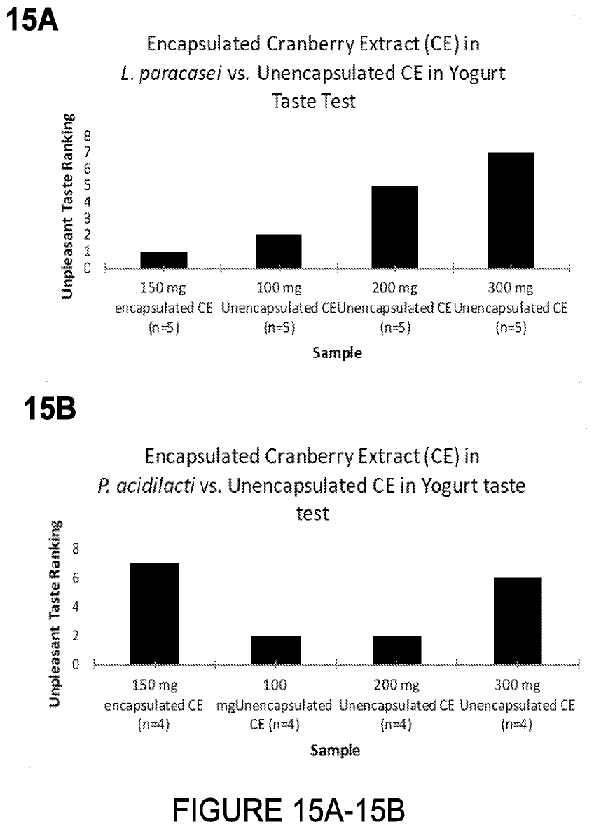

Cranberry extract is incorporated in L. paracasei such that the amount of incorporated phenolics from cranberry extract is 150 mg. Incorporated cranberry formulation (150 mg of phenolics) was mixed with 5 g of yogurt and compared with yogurt formulations having 100, 200 and 300 mg unincorporated cranberry extract mixed in 5 g of yogurt. Volunteers tasted these various yogurt formulations with incorporated cranberry extract or unincorporated cranberry extract and ranked these formulations for unpleasant taste (e.g., sour, bitter astringent, or a combination of one or more tastes) in a 0 to 10 scale. As can be seen from FIG. 15A, yogurt formulations with L. paracasei incorporated cranberry extract has significantly lower unpleasant taste as compared to yogurt formulations with unincorporated cranberry extract.

Similar experiments were repeated in which cranberry extract was incorporated in P. acidilactici. FIG. 15B shows the results of unpleasant taste masking using this formulation. Several other formulations in which cranberry extract was incorporated in L. casei, P. acidilactici, L. acidophilus and L. brevis. Cranberry extract incorporated in L. paracasei showed most masking of unpleasant taste (e.g., sour, bitter astringent, or a combination of one or more tastes).

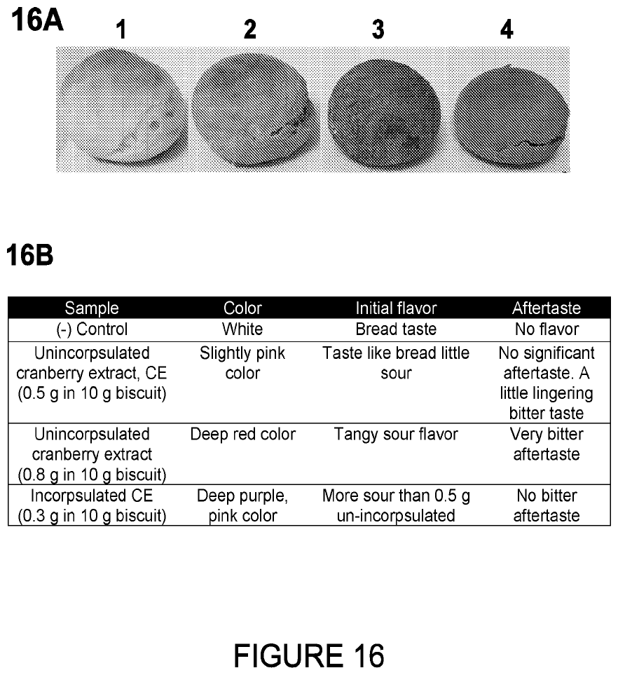

Biscuits comprising cranberry extract (CE) incorporated in L. paracasei are compared with biscuits comprising unincorporated CE for color, initial flavor, and after taste. Biscuits comprising 0.3 g cranberry extract (CE) incorporated in L. paracasei gave a deep purple color, and no bitter after taste (FIGS. 16A and B).

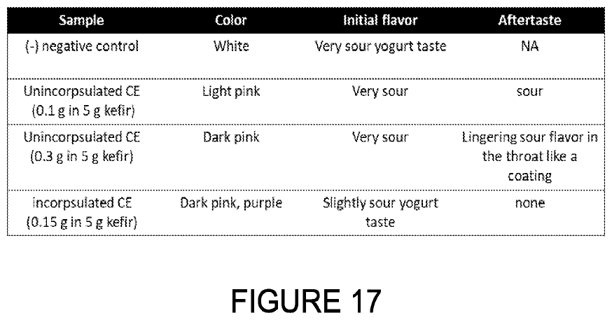

Similar experiments were tested with Kefir. 5 g of Kefir comprising 0.15 g cranberry extract (CE) incorporated in L. paracasei was compared with similar weight of Kefir comprising unincorporated C_{E} (0.1 g or 0.3 g) for color, initial flavor, and aftertaste. Kefir comprising 0.15 g cranberry extract (CE) incorporated in L. paracasei had a deep pink, purple color and no aftertaste (FIG. 17).

It was determined that there is approximately 20 mg of total phenolics per gram of unincorporated cranberry concentrate. On the other hand, there is ˜150 mg of total phenolics per gram of incorporated cranberry formulation. This large difference in total phenolic content explains the stronger color intensity of formulation with incorporated cranberry extract in comparison to the unincorporated components. This also means, in order to get the increased antioxidants with the unincorporated cranberry concentrate, one would need to add significantly larger amount of the concentrate to a finished product such as yogurt. This would drastically alter the taste profile, increasing the unpleasant taste multifold and unattractive to the consumer. On the other hand, formulations of the present application provide very high concentration of antioxidants while excluding the sugar from the fruit or vegetable concentrate.

### Example 12

### Evaluation of Viability of Cells with Incorporated Bioactive Agents

Experiments were carried out to determine the viability of the cells after various bioactive agents (e.g., cranberry extract, blackberry extract) were incorporated into the cells.

Briefly, 1 g of L. paracasei were washed twice in 10 mL distilled water to remove maltodextrin. The cells are incubated with blackberry extract in different concentrations (2M, 100 mM, and 10 mM) of citric acid-sodium citrate buffer, pH3 overnight. 6 mg of final cell slurry was added to a centrifuge tube. To the cell slurry, 1 mL 1× PBS buffer was added to prepare a stock bacterial suspension. To a 100 uL of stock bacterial suspension, a 900 uL 1× PBS buffer was added to prepare bacterial suspension of a dilution factor of 10⁻¹. This bacterial suspension was further diluted 10-fold with 1×PBS to prepare a bacterial suspension of dilution factor 10⁻². 100 uL of the bacterial suspensions were plated on MRS agar plate. The plates were incubated for 48 hours at 37° C. and the colonies were counted.

For negative control, L. paracasei cells were incubated with different concentrations (2M, 100 mM, and 10 mM) of citric acid-sodium citrate buffer, pH3 overnight without the blackberry extract. The dilutions and plating of the bacterial cells were performed as described above.

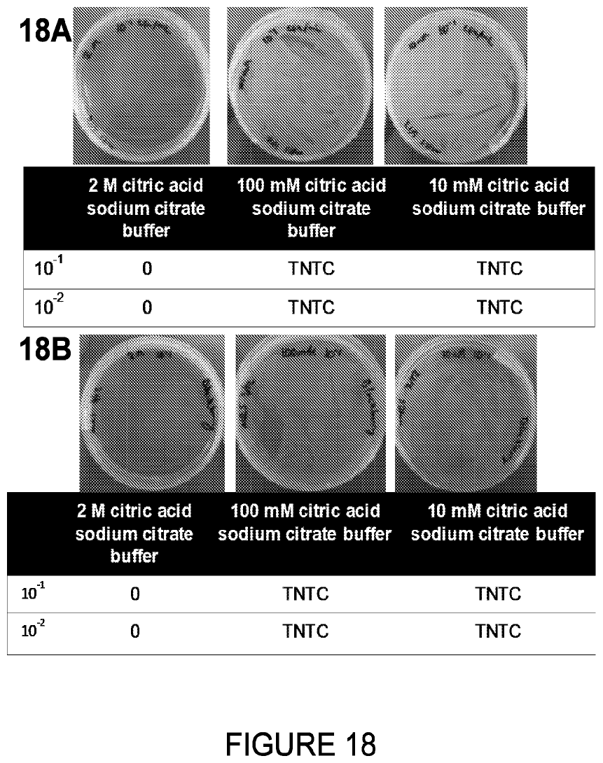

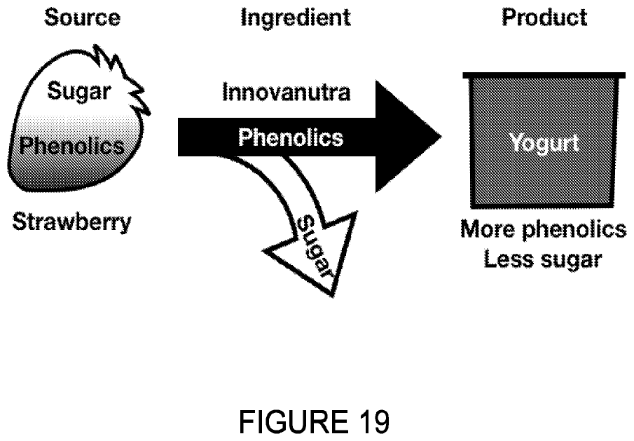

No bacterial colonies were observed when the bacterial cells were incubated overnight with 2 M citric acid-sodium citrate buffer, pH 3 in the presence or absence of blackberry extract. However, a bacterial lawn was formed with bacterial cells incubated overnight with 10 mM and 100 mM citric acid-sodium citrate buffer, pH 3 in the presence or absence of blackberry extract (FIG. 18). Thus, cells are viable when blackberry concentrate is incorporated in presence of 10 mM and 100 mM citric acid-sodium citrate buffer, pH 3.

The above experiments were repeated with cranberry concentrate. No colonies were observed when the bacterial cells were incubated with 2 M and 100 mM citric acid-sodium citrate buffer, pH 3 in the presence cranberry concentrate. However, few colonies were observed when bacterial cells were incubated with 10 mM citric acid-sodium citrate buffer, pH 3 in the presence cranberry concentrate.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred examples and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred examples, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other examples are set forth within the following claims.

1. A composition comprising:
(a) a carrier;
(b) one or more bioactive agents;
wherein said one or more bioactive agents are incorporated into said carrier, wherein said one or more bioactive agents exists in nature in their unincorporated form as a mixture with sugars, and wherein sugars from said mixture are preferentially excluded out of said carrier in said composition.

2. The composition of claim 1, wherein at least about 50% of said sugar from said mixture is excluded out of said carrier in said composition.

3.-4. (canceled)

5. The composition of claim 1, wherein said one or more bioactive agents comprise phytochemicals.

6. The composition of claim 5, wherein said phytochemical is a polyphenol, carotenoid, curcuminoid, isothiocyanate, terpenoid, lignans, phenolic acids, phytosterols, fiber, chlorophyll, or a combination thereof.

7. The composition of claim 6, wherein said polyphenol is anthocyanin, flavone, flavanone, isoflavone, flavonol, proanthocyanidin, catechin, epicatechin, procyanidin, prodelphinidin, or a combination thereof.

8. The composition of claim 1, wherein said one or more bioactive agents is a plant product, plant extract, fruit juice, fruit concentrate, vegetable extract, vegetable concentrate or a mixture thereof.

9. (canceled)

10. The composition of claim 1, wherein said sugars is a monosaccharide, disaccharide, oligosaccharides, or a mixture thereof.

11. The composition of claim 1, wherein said carrier is a cell, cell aggregates with extracellular matrix, cell aggregates without extracellular matrix, cell wall particles, an extracellular membrane of a cell, ghost cell, spores, vegetable skin particles, fruit skin particles, plant tissue, animal tissue, or a virus.

12. The composition of claim 11, wherein said carrier is a cell, and wherein said cell is either live or dead.

13. The composition of claim 9, wherein said cell is a bacterial cell.

14. (canceled)

15. The composition of claim 13, wherein the cell wall of said bacteria is modified.

16. The composition of claim 12, wherein said cell is a eukaryotic cell.

17. The composition of claim 16, wherein said cell is a yeast cell.

18. (canceled)

19. (canceled)

20. The composition of claim 11, wherein said carrier is a bacterial or a fungal spore.

21. (canceled)

22. The composition of claim 1, wherein said one or more bioactive agents incorporated into said carrier have enhanced thermal stability as compared to unincorporated bioactive agents.

23. (canceled)

24. The composition of claim 1, wherein said one or more bioactive agents incorporated into said carrier have enhanced pH stability as compared to unincorporated bioactive agents.

25. (canceled)

26. (canceled)

27. The composition of claim 1, wherein said one or more bioactive agents incorporated into said carrier have enhanced oxidative stability as compared to unincorporated bioactive agents.

28-31. (canceled)

32. The composition of claim 1, wherein said one or more bioactive agents incorporated into said carrier have enhanced color stability as compared to unincorporated bioactive agents.

33. (canceled)

34-36. (canceled)

37. The composition of claim 67, wherein the taste of said one or more bioactive agents incorporated into said carrier is masked as compared to unincorporated bioactive agents.

38-50. (canceled)

51. The composition of claim 1, wherein said carrier is a cell or a spore, and wherein said one or more bioactive agents improves the viability of the cell from exposure to heat, UV, desiccation, pH, oxidative stress, or a combination thereof.

52. The composition of claim 51, wherein said one of more bioactive agents comprise phytochemicals.

53. The composition of claim 52, wherein said phytochemical is a polyphenol.

54. The composition of claim 51, wherein said cell is a bacteria, fungal cell, spore.

55. The composition of claim 1, wherein said one or more bioactive agents are incorporated into said carrier in the presence of 0.005 M to 4 M of citric acid-sodium citrate at a pH of about 3-6.

56. (canceled)

57. The composition of claim 11, wherein said carrier is cell aggregate, and wherein the diameter of said cell aggregate is in the range of about 0.01 mm to about 5 mm.

58.-66. (canceled)

67. The composition of claim 1, wherein said composition is a liquid, solid, gel or foam.

68. The composition of claim 1, wherein said one or more bioactive agents incorporated into said carrier can be extracted from said carrier in the presence of 5-100% ethanol, dimethyl sulfoxide, 5-100% methanol, acidified methanol and 0.005 M to 4 M citric acid-sodium citrate buffer at a pH of about 3-6.
